SEQUENCE LISTING (48 sequences)

SEQ ID NO: 1 - protein, 1294 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDEYK

SEQ ID NO: 2 - protein, 792 aa, synthetic construct
MALPVTALLLPLALLLHAARPMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 3 - protein, 792 aa, synthetic construct
MALPVTALLLPLALLLHAARPMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 4 - protein, 791 aa, synthetic construct
MALPVTALLLPLALLLHAARPMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 5 - protein, 791 aa, synthetic construct
MALPVTALLLPLALLLHAARPMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 6 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 7 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 8 - protein, 252 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 9 - protein, 121 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 10 - protein, 108 aa, synthetic construct
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 11 - protein, 107 aa, synthetic construct
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 12 - protein, 286 aa, synthetic construct
MKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSSGGGGSGGGGSGGGGSGGGGSMMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIK

SEQ ID NO: 13 - protein, 5 aa, synthetic construct
NYRMH

SEQ ID NO: 14 - protein, 17 aa, synthetic construct
GIYPGNSDTNYNQKFKD

SEQ ID NO: 15 - protein, 9 aa, synthetic construct
GIRGSYFDY

SEQ ID NO: 16 - protein, 15 aa, synthetic construct
KASQSVDYDGDSYMN

SEQ ID NO: 17 - protein, 7 aa, synthetic construct
AASNLES

SEQ ID NO: 18 - protein, 9 aa, synthetic construct
QQSNEDPST

SEQ ID NO: 19 - protein, 137 aa, synthetic construct
MECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSS

SEQ ID NO: 20 - protein, 131 aa, synthetic construct
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVK

SEQ ID NO: 21 - protein, 288 aa, synthetic construct
MECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVK

SEQ ID NO: 22 - protein, 288 aa, synthetic construct
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSS

SEQ ID NO: 23 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 24 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 25 - protein, 42 aa, synthetic construct
KRGRKKLLYIFKQPFMRPVQTTQEEDGCSCRFPEEEEGGCEL

SEQ ID NO: 26 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 112 aa, synthetic construct; identical to SEQ ID NO: 26

SEQ ID NO: 28 - protein, 41 aa, synthetic construct
RSKRSRLLHSDYMNMTPRRPGPTRKHYQPYAPPRDFAAYRS

SEQ ID NO: 29 - protein, 21 aa, synthetic construct
MALPVTALLLPLALLLHAARP

SEQ ID NO: 30 - protein, 21 aa, synthetic construct
METDTLLLWVLLLWVPGSTGD

SEQ ID NO: 31 - protein, 12 aa, synthetic construct
GGGSGGGSGGGS

SEQ ID NO: 32 - protein, 15 aa, synthetic construct
GGGGSGGGGSGGGGS

SEQ ID NO: 33 - protein, 20 aa, synthetic construct
GGGGSGGGGSGGGGSGGGGS

SEQ ID NO: 34 - protein, 18 aa, synthetic construct
GSTSGSGKPGSGEGSTKG

SEQ ID NO: 35 - protein, 18 aa, synthetic construct
GGSSRSSSSGGGGSGGGG

SEQ ID NO: 36 - protein, 24 aa, synthetic construct
SGGGGEGRGSLLTCGDVEENPGPR

SEQ ID NO: 37 - protein, 250 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 38 - protein, 69 aa, synthetic construct; identical to SEQ ID NO: 24

SEQ ID NO: 39 - protein, 6 aa, synthetic construct
HHHHHH

SEQ ID NO: 40 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 41 - protein, 105 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEI

SEQ ID NO: 42 - protein, 10 aa, synthetic construct
GYSFTGYTMN

SEQ ID NO: 43 - protein, 17 aa, synthetic construct
LITPYNGASSYNQKFRG

SEQ ID NO: 44 - protein, 10 aa, synthetic construct
GGYDGRGFDY

SEQ ID NO: 45 - protein, 10 aa, synthetic construct
SASSSVSYMH

SEQ ID NO: 46 - protein, 7 aa, synthetic construct
DTSKLAS

SEQ ID NO: 47 - protein, 9 aa, synthetic construct
QQWSGYPLT

SEQ ID NO: 48 - protein, 5 aa, synthetic construct
GGGGS

(19) United States
(12) Patent Application Publication
(10) Pub. No.: US 2025/0000978 A1
(43) Pub. Date: Jan. 2, 2025

(54) ANTI-MESOTHELIN CAR T CELLS SECRETING TEAMS AND METHODS OF USE THEREOF

(71) Applicant: THE GENERAL HOSPITAL CORPORATION, Boston, MA (US)

(72) Inventors: Marcela V. Maus, Lexington, MA (US); Marc Wehrli, Charlestown, MA (US)

(73) Assignee: THE GENERAL HOSPITAL CORPORATION, Boston, MA (US)

(21) Appl. No.: 18/707,459

(22) PCT Filed: Nov. 4, 2022
(86) PCT No.: PCT/US2022/079284

§ 371 Date: May 3, 2024

Related U.S. Application Data
(60) Provisional application No. 63/278,825, filed on Nov. 12, 2021.
(60) Provisional application No. 63/275,911, filed on Nov. 4, 2021.

(51) Int. Cl. A61K 39/00; A61P 35/00; C07K 16/28

(52) U.S. Cl. CPC A61K 39/464468; A61K 39/4611; A61K 39/4631; A61P 35/00; C07K 16/2803; C07K 2317/622

(57) ABSTRACT

The present disclosure relates to mesothelin chimeric antigen receptors (CARs), T cell engaging molecules (TEAMs), anti-mesothelin-CAR T cells optionally comprising TEAMs, and methods of use thereof.

### RELATED APPLICATION

This application claims the benefit under 35 U.S.C. § 119 (e) to U.S. Provisional Application No. 63/275,911, filed Nov. 4, 2021, entitled “Anti-mesothelin CAR T cells secreting TEAMs and Methods of Use Thereof”, and U.S. Provisional Application No. 63/278,825, filed Nov. 12, 2021, entitled “Anti-mesothelin CAR T cells secreting TEAMs and Methods of Use Thereof”, the entire contents of each of which are incorporated herein by reference.

### FEDERALLY SPONSORED RESEARCH

This invention was made with Government support under R01 CA 238268, awarded by the National Cancer Institute, and T32CA009216, awarded by the National Institute of Health. The Government has certain rights in the invention.

### REFERENCE TO AN ELECTRONIC SEQUENCE LISTING

The content of the electronic sequence listing (M105370026WO00-SEQ-ARM.xml; Size: 50,238 bytes; and Date of Creation: Nov. 3, 2022) is herein incorporated by reference in its entirety.

### BACKGROUND

Numerous diseases, including cancer and autoimmune diseases, are associated with mesothelin over-expression. Chimeric antigen receptor (CAR) T cells have been used previously to specifically bind to diseased cells, which resulted in killing of the diseased cell.

### SUMMARY

Described herein are CARs and CAR T cells that target mesothelin, which can be useful for treating a subject having a disease or disorder, e.g., a cancer, a plasma cell disease or disorder, or an autoimmune disease or disorder. Also provided herein are nucleic acids and vectors encoding said CARs, and cells comprising the CARs, nucleic acids, or vectors described herein. Also provided herein are pharmaceutical compositions comprising CARs, CAR T cells, and other compositions described herein. Further provided are methods of treating cancer or an autoimmune disease in a subject by administering a composition described herein.

Accordingly, in one aspect, the disclosure is directed to a chimeric antigen receptor (CAR) T cell comprising a heterologous nucleic acid molecule, wherein the heterologous nucleic acid molecule comprises a first polynucleotide encoding a CAR comprising: an extracellular antigen-binding domain that binds to a tumor antigen comprising mesothelin or a portion thereof; a transmembrane domain; and an intracellular signaling domain.

In some embodiments, the CAR T cell further comprises a second polynucleotide encoding a therapeutic agent. In some embodiments, the second polynucleotide is part of the heterologous nucleic acid molecule. In some embodiments, the CAR T cell further comprises a second heterologous nucleic acid molecule comprising the second polynucleotide.

In another aspect, the disclosure is directed to a chimeric antigen receptor (CAR) comprising: an extracellular antigen-binding domain that binds to a tumor antigen comprising mesothelin or a portion thereof, a transmembrane domain, and an intracellular signaling domain. In some embodiments, the CAR comprises a polynucleotide or polypeptide therapeutic agent.

In some embodiments, the therapeutic agent comprises an antibody reagent. In some embodiments, the antibody reagent comprises a single chain antibody or a single domain antibody. In some embodiments, the antibody reagent comprises a bispecific antibody reagent. In some embodiments, the bispecific antibody reagent comprises a T cell engaging antibody molecule (TEAM). In some embodiments, the single domain antibody comprises a camelid antibody. In some embodiments, the therapeutic agent comprises a cytokine.

In some embodiments, the CAR and the therapeutic agent are produced in the form of a single polypeptide, which is cleaved to generate separate CAR and therapeutic agent molecules. In some embodiments, the single polypeptide comprises a cleavable moiety between the CAR and the therapeutic agent. In some embodiments, the cleavable moiety comprises: a 2A peptide, a 2A ribosomal skip sequence, or an IRES. In some embodiments, the 2A peptide comprises P2A or T2A.

In some embodiments, the CAR and the therapeutic agent are each constitutively expressed. In some embodiments, expression of the CAR and the therapeutic agent is driven by an elongation factor-1 alpha (EF1α) promoter. In some embodiments, the therapeutic agent is expressed under the control of an inducible promoter, which is optionally inducible by T cell receptor or CAR signaling. In some embodiments, the inducible promoter comprises the NFAT promoter. In some embodiments, the CAR is expressed under the control of a constitutive promoter and the therapeutic agent is expressed under the control of an inducible promoter, which is optionally inducible by T cell receptor or CAR signaling. In some embodiments, the CAR expressed from a first vector and the therapeutic agent is expressed from a second vector.

In some embodiments, the CAR comprises one or more co-stimulatory domains. In some embodiments, the co-stimulatory domain comprises a 4-1BB co-stimulatory domain, which optionally comprises the sequence of SEQ ID NO: 25 or a variant thereof.

In some embodiments, the antigen-binding domain of the CAR comprises an antibody, a single chain antibody, a single domain antibody, or a ligand.

In some embodiments, the transmembrane domain of the CAR comprises a CD8 hinge/transmembrane domain, which optionally comprises the sequence of SEQ ID NO: 24, or a variant thereof. In some embodiments, the intracellular signaling domain comprises a CD3 intracellular signaling domain, which optionally comprises the sequence of any one of SEQ ID NOs: 26-27, or a variant thereof.

In some embodiments, the therapeutic agent binds to a tumor antigen. In some embodiments, the tumor antigen to which the therapeutic agent binds is a solid tumor antigen. In some embodiments, the tumor antigen to which the therapeutic agent binds is an activated fibroblast marker. In some embodiments, the activated fibroblast marker comprises: aSMA (ACTA2), fibroblast activation protein (FAP), platelet derived growth factor receptor-α and -β(PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1). In some embodiments, the activated fibroblast marker comprises FAP.

In some embodiments, the VH of the tumor antigen binding domain of the antibody reagent comprises an amino acid sequence of SEQ ID NO: 6, or an amino acid sequence having at least 90% sequence identity to the amino acid sequence of SEQ ID NO: 6. In some embodiments, the VL of the tumor antigen binding domain of the antibody reagent comprises an amino acid sequence of SEQ ID NO: 7, or an amino acid sequence having at least 90% sequence identity to the amino acid sequence of SEQ ID NO: 7. In some embodiments, the tumor antigen binding domain of the antibody reagent comprises an amino acid sequence of SEQ ID NO: 8, or an amino acid sequence having at least 90% sequence identity to the amino acid sequence of SEQ ID NO: 8. In some embodiments, the VH of the tumor antigen binding domain of the antibody reagent is N-terminal to the VL of the tumor antigen binding domain of the antibody reagent. In some embodiments, the VL of the tumor antigen binding domain of the antibody reagent is N-terminal to the VH of the tumor antigen binding domain of the antibody reagent. In some embodiments, the tumor antigen binding domain of the antibody reagent comprises a single-chain variable fragment (scFv) or a single domain antibody, which optionally comprises a sequence of SEQ ID NO: 8, or a variant thereof.

In some embodiments, the antibody reagent binds to a T cell surface antigen. In some embodiments, the T cell surface antigen comprises T cell receptor (TCR), a TCR complex component, or a portion of either thereof. In some embodiments, the TCR complex component is chosen from CD3 (e.g., the CD3εsubunit, the CD3ζ subunit, or the CD3γ subunit), the CD4 coreceptor, and the CD8 coreceptor. In some embodiments, the TCR complex component is CD3.

In some embodiments, the VH of the T cell surface antigen-binding domain of the antibody reagent comprises an amino acid sequence of SEQ ID NO: 9, or an amino acid sequence having at least 90% sequence identity to the amino acid sequence of SEQ ID NO: 9. In some embodiments, the VL of the T cell surface antigen-binding domain of the antibody reagent comprises an amino acid sequence of SEQ ID NO: 10, or an amino acid sequence having at least 90% sequence identity to the amino acid sequence of SEQ ID NO: 10. In some embodiments, the VH of the T cell surface antigen-binding domain of the antibody reagent is N-terminal to the VL of the T cell surface antigen-binding domain of the antibody reagent. In some embodiments, the VL of the T cell surface antigen-binding domain of the antibody reagent is N-terminal to the VH of the T cell surface antigen-binding domain of the antibody reagent. In some embodiments, the T cell surface antigen-binding domain of the antibody reagent comprises a scFv or a single domain antibody, which optionally comprises a sequence of any one of SEQ ID NOs: 11-12, or a variant thereof.

In some embodiments, the antigen-binding domain of the CAR comprises: (a) a heavy chain variable domain (VH) comprising three complementarity determining regions CDR-H1, CDR-H2, and CDR-H3, wherein the CDR-H1 comprises an amino acid sequence of SEQ ID NO: 13, or an amino acid sequence with no more than 1, 2, or 3 amino acid substitutions of SEQ ID NO: 13; the CDR-H2 comprises an amino acid sequence of SEQ ID NO: 14, or an amino acid sequence with no more than 1, 2, or 3 amino acid substitutions of SEQ ID NO: 14; and the CDR-H3 comprises an amino acid sequence of SEQ ID NO: 15, or an amino acid sequence with no more than 1, 2, or 3 amino acid substitutions of SEQ ID NO: 15, and (b) a light chain variable domain (VL) comprising three complementarity determining regions CDR-L1, CDR-L2, and CDR-L3, wherein the CDR-L1 comprises an amino acid sequence of SEQ ID NO: 16, or an amino acid sequence with no more than 1, 2, or 3 amino acid substitutions of SEQ ID NO: 16; the CDR-L2 comprises an amino acid sequence of SEQ ID NO: 17, or an amino acid sequence with no more than 1, 2, or 3 amino acid substitutions of SEQ ID NO: 17; and the CDR-L3 comprises an amino acid sequence of SEQ ID NO: 18, or an amino acid sequence with no more than 1, 2, or 3 amino acid substitutions of SEQ ID NO: 18. In some embodiments, the VH of the antigen-binding domain of the CAR comprises an amino acid sequence of SEQ ID NO: 19, or an amino acid sequence having at least 90% sequence identity to the amino acid sequence of SEQ ID NO: 19. In some embodiments, the VL of the antigen-binding domain of the CAR comprises an amino acid sequence of SEQ ID NO: 20, or an amino acid sequence having at least 90% sequence identity to the amino acid sequence of SEQ ID NO: 20. In some embodiments, the VH of the antigen-binding domain of the CAR is N-terminal to the VL of the antigen-binding domain of the CAR. In some embodiments, the VL of the antigen-binding domain of the CAR is N-terminal to the VH of the antigen-binding domain of the CAR. In some embodiments, the antigen-binding domain of the CAR comprises a scFv or a single domain antibody, which optionally comprises a sequence selected from the group consisting of SEQ ID NOs: 21 or 22, and variants thereof.

In some embodiments, the antigen-binding domain of the CAR comprises: (a) a heavy chain variable domain (VH) comprising complementarity determining regions CDR-H1, CDR-H2, and CDR-H3, wherein the CDR-H1 comprises the amino acid sequence of SEQ ID NO: 42, or an amino acid sequence with no more than 1, 2, or 3 amino acid substitutions relative thereto; the CDR-H2 comprises an amino acid sequence of SEQ ID NO: 43, or an amino acid sequence with no more than 1, 2, or 3 amino acid substitutions relative thereto; and the CDR-H3 comprises an amino acid sequence of SEQ ID NO: 44, or an amino acid sequence with no more than 1, 2, or 3 amino acid substitutions relative thereto, and (b) a light chain variable domain (VL) comprising complementarity determining regions CDR-L1, CDR-L2, and CDR-L3, wherein the CDR-L1 comprises the amino acid sequence of SEQ ID NO: 45, or an amino acid sequence with no more than 1, 2, or 3 amino acid substitutions relative thereto; the CDR-L2 comprises an amino acid sequence of SEQ ID NO: 46, or an amino acid sequence with no more than 1, 2, or 3 amino acid substitutions relative thereto; and the CDR-L3 comprises an amino acid sequence of SEQ ID NO: 47, or an amino acid sequence with no more than 1, 2, or 3 amino acid substitutions relative thereto. In some embodiments, the VH of the antigen-binding domain of the CAR comprises the SS1 heavy chain sequence of SEQ ID NO: 40, or an amino acid sequence having at least 90% sequence identity to the SS1 heavy chain sequence of SEQ ID NO: 40. In some embodiments, the VL of the antigen-binding domain of the CAR comprises the SS1 light chain sequence of SEQ ID NO: 41, or an amino acid sequence having at least 90% sequence identity to the SS1 light chain sequence of SEQ ID NO: 41. In some embodiments, the VH of the antigen-binding domain of the CAR is N-terminal to the VL of the antigen-binding domain of the CAR. In some embodiments, the VL of the antigen-binding domain of the CAR is N-terminal to the VH of the antigen-binding domain of the CAR. In some embodiments, the antigen-binding domain of the CAR comprises a scFv or a single domain antibody, which optionally comprises the SS1 VH amino acid sequence of SEQ ID NO: 40 and the SS1 VL amino acid sequence of SEQ ID NO: 41, and variants thereof.

In some embodiments, the heterologous nucleic acid molecule further comprises a suicide gene.

In another aspect, the disclosure is directed to a nucleic acid molecule comprising a sequence encoding a CAR polypeptide described herein. In some embodiments, the nucleic acid molecule further comprises a sequence encoding a therapeutic agent described herein. In some embodiments, the nucleic acid molecule is a plasmid or a vector.

In another aspect, the disclosure is directed to a viral vector comprising a nucleic acid molecule described herein. In some embodiments, the viral vector is a lentiviral or adeno-associated viral vector.

In another aspect, the disclosure is directed to a pharmaceutical composition comprising a CAR T cell, a CAR polypeptide, or a nucleic acid molecule or viral vector described herein. In some embodiments, the pharmaceutical composition comprises a therapeutically effective amount of a CAR T cell, a CAR polypeptide, or a nucleic acid molecule or viral vector described herein. In some embodiments, the pharmaceutical composition comprises a pharmaceutically acceptable excipient.

In another aspect, the disclosure is directed to a method of treating a patient having cancer, the method comprising administering to the patient a pharmaceutical composition described herein. In some embodiments, by targeting the tumor microenvironment, systemic toxicity is reduced. In some embodiments, the cancer is characterized by the presence of one or more solid tumors. In some embodiments, the cancer is characterized by expression of mesothelin. In some embodiments, the tumor microenvironment is characterized by the presence of activated fibroblasts. In some embodiments, the tumor microenvironment is characterized by the presence of fibroblast activating protein (FAP). In some embodiments, the cancer is a pancreatic cancer, a lung cancer, an ovarian cancer, endometrial cancer, biliary cancer, gastric cancer, or mesothelioma.

In another aspect, the disclosure is directed to a method of treating a patient having cancer, the method comprising administering to the patient a CAR T cell product, genetically modified to secrete a tumor-toxic antibody or cytokine, wherein by directing the cancer toxicity locally to the tumor microenvironment, systemic toxicity is reduced. In some embodiments, the CAR T cell is genetically modified to deliver an antibody or a bispecific antibody against an activated fibroblast marker to the tumor microenvironment. In some embodiments, the CAR T cell comprises a polynucleotide encoding a CAR comprising an extracellular antigen-binding domain that binds to a tumor antigen comprising mesothelin or a portion thereof. In some embodiments, the bispecific antibody is directed against an activated fibroblast marker and CD3.

In another aspect, the disclosure is directed to a method of delivering a therapeutic agent to a tissue or organ in a patient to treat a disease or pathology, the method comprising administering to said patient a CAR T cell, genetically modified to secrete a therapeutic antibody, toxin, or agent, wherein the therapeutic antibody, toxin, or agent would, by itself, be unable to enter or penetrate the tissue or organ. In some embodiments, the tissue or organ is in the digestive or endocrine system. In some embodiments, the tissue is in the pancreas or the organ is the pancreas. In some embodiments, the tissue or organ is in the reproductive system. In some embodiments, the tissue is in an ovary or the organ is an ovary. In some embodiments, the tissue or organ is in the respiratory or pulmonary system. In some embodiments, the tissue is in a lung or the organ is a lung. In some embodiments, the therapeutic antibody is directed against an activated fibroblast marker.

In some embodiments of a method described herein, the CAR T cell is a CAR T cell described herein, or the CAR T cell is comprised within a pharmaceutical composition described herein.

For convenience, the meaning of some terms and phrases used in the specification, examples, and appended claims, are provided below. Unless stated otherwise, or implicit from context, the following terms and phrases include the meanings provided below. The meanings are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its meaning provided herein, the meaning provided within the specification shall prevail.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. A subject can be one who has been previously diagnosed with or identified as suffering from or having a condition in need of treatment (e.g., mesothelioma, or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject 20) can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “tumor antigen” and “cancer antigen” are used interchangeably to refer to antigens that are differentially expressed by cancer cells and can thereby be exploited in order to target cancer cells (e.g. mesothelin). Cancer antigens are antigens that can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated Ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), human epidermal growth factor receptor (HER2), mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. In some embodiments, the tumor antigen is mesothelin. Examples of tumor antigens are provided below and include, e.g., mesothelin, EGFR, EGFRVIII, CD19, PSMA, B cell maturation antigen (BCMA), interleukin-13 receptor subunit alpha-2 (IL13Ra2), etc. In some embodiments, a tumor antigen is a tumor-associated antigen. A tumor-associated antigen can be present proximal to cancer cells or a tumor, but not necessarily attached to the cancer cell or a tumor. For example, a tumor-associated antigen may be a marker present (e.g., at an increased level relative to a normal tissue) in the extracellular matrix surrounding or adjacent to a tumor.

As used herein, the term “chimeric” refers to the product of the fusion of portions of at least two or more different polynucleotide molecules. In some embodiments, the term “chimeric” refers to a gene expression element produced through the manipulation of known elements or other polynucleotide molecules.

As used herein, the term “T cell engaging antibody molecules” or “TEAMs” is meant polypeptides that each include tandemly linked single-chain variable fragments (scFvs). Optionally, the scFvs are linked by a linker (e.g., a glycine-rich linker). One scFv of the TEAM binds to a T cell surface antigen (e.g., the T cell receptor (TCR) or a portion thereof (e.g., to the CD3ε subunit)) and the other binds to a target antigen (e.g., a tumor antigen). In some embodiments, the T cell surface antigen is a Treg antigen.

As used herein, “Treg antigen” or “Treg-associated antigen” is used interchangeably to refer to antigens that are expressed by T regulatory (Treg) cells. These antigens may optionally be targeted by the antibody reagents, antibody conjugates, and methods of the disclosure. Examples of Treg antigens are provided below and include, e.g., GARP, LAP, CD25, and CTLA-4.

In some embodiments, “activation” can refer to the state of a T cell that has been sufficiently stimulated to induce detectable cellular proliferation. In some embodiments, activation can refer to induced cytokine production. In other embodiments, activation can refer to detectable effector functions.

At a minimum, an “activated T cell” as used herein is a proliferative T cell.

As used herein, the term “therapeutically effective amount” refers to the amount necessary or sufficient to realize a desired biologic effect. For example, a therapeutically effective amount of a CAR, CAR T cell, or antibody reagent of the present disclosure may be that amount sufficient to ameliorate one or more symptoms of a cancer. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the present disclosure without necessitating undue experimentation.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC) (e.g., a macrophage, a dendritic cell, a B-cell, an artificial APC, and the like) can specifically bind with a cognate binding partner (referred to herein as a “stimulatory molecule” or “costimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an antiCD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell. “Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1BBL, OX40L, CD7, B7-1 (CD80), B7-2 (CD86), PD-L1, PD-L2, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, IL T3, IL T4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule, BTLA, a Toll-like receptor, CD27, CD28, 4-1BB, OX40, CD30, CD40, PD-1, ICOS, lymphocyte function associated antigen-1 (LFA-1), CD2, CD7, LIGHT, NKG2C, B7-H3, and CD83.

In some embodiments, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene.

The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

The term “polynucleotide” is used herein interchangeably with “nucleic acid molecule” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. In some embodiments, the nucleic acid molecule is a heterologous nucleic acid molecule. As used herein the term, “heterologous nucleic acid molecule” refers to a nucleic acid molecule that does not naturally exist within a given cell.

A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g., a CAR polypeptide) is comprised by a vector. In some of the aspects described herein, a nucleic acid sequence encoding a given polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′ UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, a “signal peptide” or “signal sequence” refers to a peptide at the N-terminus of a newly synthesized protein that serves to direct a nascent protein into the endoplasmic reticulum. In some embodiments, the signal peptide is a CD8 or IgK signal peptide.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g., cancer or an autoimmune disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the terms “about” or “approximately” when used in connection with a quantity refer to a range of values around said quantity of ±1, 2, 3, 4, 5, 6, 7, 8, 9, or 10%, e.g., ±1% or ±10%.

As used herein, the singular terms “a,” “an,” and “the” include plural referents unless context clearly indicates otherwise. Similarly, the word “or” is intended to include “and” unless the context clearly indicates otherwise. Other terms are defined within the description of the various aspects and embodiments of the technology, as set forth below.

### BRIEF DESCRIPTION OF THE DRAWINGS

The accompanying drawings are not intended to be drawn to scale. The patent or application file contains at least one drawing executed in color. Copies of this patent or patent application publication with color drawing(s) will be provided by the Office upon request and payment of the necessary fee.

FIGS. 1A-1E are graphs showing cytokine production in SS1 anti-mesothelin CAR T cells expressing a previously known anti-mesothelin SS1 CAR compared to exemplary CAR T cells of the disclosure expressing an MGHmesol L/H anti-mesothelin CAR of the disclosure (untransduced control in white). FIG. 1F shows graphs of cell lysis assessed in an overnight luciferase-based killing assay with Mesothelin+ pancreatic cancer (Capan-2) (at top) or Mesothelin-lymphoma cells (JeKo-1) cell lines (at bottom).

FIGS. 2A-2B show an overview of the experimental mouse protocol used to assess the efficacy of anti-mesothelin SS1 CAR or anti-mesothelin MGHmesol L/H CAR. FIG. 2A shows the schedule of administering pancreatic cancer cells (AsPC-1 cells) to mice and subsequent administration of CAR T cells. FIG. 2B shows the CAR T dosing amounts, treatment days, and number of mice per treatment.

FIGS. 3A-3D compare anti-mesothelin CAR T cells with a previously known anti-mesothelin SS1 CAR (C) or the disclosed anti-mesothelin MGHmesol L/H CAR (B) in mice injected with ASPC1 pancreatic cancer cells (no CAR T cell injection in gray). 1×10⁶ (1e6) CAR T cells were administered in each experiment. Each of FIGS. 3A-3D have a panel showing flux and survival curves associated with administration of the CAR-T dose in the FIG. header. FIG. 3A compares SS1 CAR T cells and MGHmesol L/H CAR T cells administered intravenously (IV) 7 days after injection with cancer cells. FIG. 3B compares SS1 CAR T cells and MGHmesol L/H CAR T cells administered by IV 14 days after injection with cancer cells. FIG. 3C compares SS1 CAR T cells and MGHmesol L/H CAR T cells administered by intraperitoneal (IP) injection 7 days after injection with cancer cells. FIG. 3D compares SS1 CAR T cells and MGHmesol L/H CAR T cells administered by intraperitoneal (IP) injection 14 days after injection with cancer cells.

FIGS. 4A-4D compare anti-mesothelin CAR T cells with a previously known anti-mesothelin SS1 CAR (C) or the disclosed anti-mesothelin MGHmesol L/H CAR (B) in mice injected with ASPC1 pancreatic cancer cells (no CAR T cell injection in gray). 2×10⁶ (2e6) CAR T cells were administered in each experiment. Each of FIGS. 4A-4D have a panel showing flux and survival curves associated with administration of the CAR-T dose in the FIG. header. FIG. 4A compares SS1 CAR T cells and MGHmesol L/H CAR T cells administered intravenously (IV) 7 days after injection with cancer cells. FIG. 4B compares SS1 CAR T cells and MGHmesol L/H CAR T cells administered by IV 14 days after injection with cancer cells. FIG. 4C compares SS1 CAR T cells and MGHmesol L/H CAR T cells administered by intraperitoneal (IP) injection 7 days after injection with cancer cells. FIG. 4D compares SS1 CAR T cells and MGHmesol L/H CAR T cells administered by intraperitoneal (IP) injection 14 days after injection with cancer cells.

FIGS. 5A-5D compare anti-mesothelin CAR T cells with a previously known anti-mesothelin SS1 CAR (C) or the disclosed anti-mesothelin MGHmesol L/H CAR (B) in mice injected with BXPC3 pancreatic cancer cells (no CAR T cell injection in gray). 1×10⁶ (1e6) CAR T cells were administered in each experiment. Each of FIGS. 5A-5D have a panel showing flux and survival curves associated with administration of the CAR-T dose in the FIG. header. FIG. 5A compares SS1 CAR T cells and MGHmesol L/H CAR T cells administered intravenously (IV) 7 days after injection with cancer cells. FIG. 5B compares SS1 CAR T cells and MGHmesol L/H CAR T cells administered by IV 14 days after injection with cancer cells. FIG. 5C compares SS1 CAR T cells and MGHmesol L/H CAR T cells administered by intraperitoneal (IP) injection 7 days after injection with cancer cells. FIG. 5D compares SS1 CAR T cells and MGHmesol L/H CAR T cells administered by intraperitoneal (IP) injection 14 days after injection with cancer cells.

FIGS. 6A-6D compare anti-mesothelin CAR T cells with a previously known anti-mesothelin SS1 CAR (C) or the disclosed anti-mesothelin MGHmesol L/H CAR (B) in mice injected with BXPC3 pancreatic cancer cells (no CAR T cell injection in gray). 2×10⁶ (2e6) CAR T cells were administered in each experiment. Each of FIGS. 6A-6D have a panel showing flux and survival curves associated with administration of the CAR-T dose in the FIG. header. FIG. 6A compares SS1 CAR T cells and MGHmesol L/H CAR T cells administered intravenously (IV) 7 days after injection with cancer cells. FIG. 6B compares SS1 CAR T cells and MGHmesol L/H CAR T cells administered by IV 14 days after injection with cancer cells. FIG. 6C compares SS1 CAR T cells and MGHmesol L/H CAR T cells administered by intraperitoneal (IP) injection 7 days after injection with cancer cells. FIG. 6D compares SS1 CAR T cells and MGHmesol L/H CAR T cells administered by intraperitoneal (IP) injection 14 days after injection with cancer cells.

FIGS. 7A-7D compare anti-mesothelin CAR T cells with a previously known anti-mesothelin SS1 CAR (C) or the disclosed anti-mesothelin MGHmesol L/H CAR (B) in mice injected with PANC1 pancreatic cancer cells (no CAR T cell injection in gray). 1×10⁶ (1e6) CAR T cells were administered in each experiment. Each of FIGS. 7A-7D have a panel showing flux and survival curves associated with administration of the CAR-T dose in the FIG. header. FIG. 7A compares SS1 CAR T cells and MGHmesol L/H CAR T cells administered intravenously (IV) 7 days after injection with cancer cells. FIG. 7B compares SS1 CAR T cells and MGHmesol L/H CAR T cells administered by IV 14 days after injection with cancer cells. FIG. 7C compares SS1 CAR T cells and MGHmesol L/H CAR T cells administered by intraperitoneal (IP) injection 7 days after injection with cancer cells. FIG. 7D compares SS1 CAR T cells and MGHmesol L/H CAR T cells administered by intraperitoneal (IP) injection 14 days after injection with cancer cells.

FIGS. 8A-8D compare anti-mesothelin CAR T cells with a previously known anti-mesothelin SS1 CAR (C) or the disclosed anti-mesothelin MGHmesol L/H CAR (B) in mice injected with PANC1 pancreatic cancer cells (no CAR T cell injection in gray). 2×10⁶ (1e6) CAR T cells were administered in each experiment. Each of FIGS. 8A-8D have a panel showing flux and survival curves associated with administration of the CAR-T dose in the FIG. header. FIG. 8A compares SS1 CAR T cells and MGHmesol L/H CAR T cells administered intravenously (IV) 7 days after injection with cancer cells. FIG. 8B compares SS1 CAR T cells and MGHmesol L/H CAR T cells administered by IV 14 days after injection with cancer cells. FIG. 8C compares SS1 CAR T cells and MGHmesol L/H CAR T cells administered by intraperitoneal (IP) injection 7 days after injection with cancer cells. FIG. 8D compares SS1 CAR T cells and MGHmesol L/H CAR T cells administered by intraperitoneal (IP) injection 14 days after injection with cancer cells.

FIGS. 9A-9D compare anti-mesothelin CAR T cells with a previously known anti-mesothelin SS1 CAR (C) or the disclosed anti-mesothelin MGHmesol L/H CAR (B) in mice injected with CAPAN2 pancreatic cancer cells (no CAR T cell injection in gray). 2.5×10⁵ (2.5e5) CAR T cells were administered in each experiment. Each of FIGS. 9A-9D have a panel showing flux and survival curves associated with administration of the CAR-T dose in the FIG. header. FIG. 9A compares SS1 CAR T cells and MGHmesol L/H CAR T cells administered intravenously (IV) 7 days after injection with cancer cells. FIG. 9B compares SS1 CAR T cells and MGHmesol L/H CAR T cells administered by IV 14 days after injection with cancer cells. FIG. 9C compares SS1 CAR T cells and MGHmesol L/H CAR T cells administered by intraperitoneal (IP) injection 7 days after injection with cancer cells. FIG. 9D compares SS1 CAR T cells and MGHmesol L/H CAR T cells administered by intraperitoneal (IP) injection 14 days after injection with cancer cells.

FIGS. 10A-10D compare anti-mesothelin CAR T cells with a previously known anti-mesothelin SS1 CAR (C) or the disclosed anti-mesothelin MGHmesol L/H CAR (B) in mice injected with CAPAN2 pancreatic cancer cells (no CAR T cell injection in gray). 5×10⁵ (5e5) CAR T cells were administered in each experiment. Each of FIGS. 10A-10D have a panel showing flux and survival curves associated with administration of the CAR-T dose in the FIG. header. FIG. 10A compares SS1 CAR T cells and MGHmesol L/H CAR T cells administered intravenously (IV) 7 days after injection with cancer cells. FIG. 10B compares SS1 CAR T cells and MGHmesol L/H CAR T cells administered by IV 14 days after injection with cancer cells. FIG. 10C compares SS1 CAR T cells and MGHmesol L/H CAR T cells administered by intraperitoneal (IP) injection 7 days after injection with cancer cells. FIG. 10D compares SS1 CAR T cells and MGHmesol L/H CAR T cells administered by intraperitoneal (IP) injection 14 days after injection with cancer cells.

FIGS. 11A-11C compare anti-mesothelin CAR T cells with CARs comprising a 41 BBz intracellular signaling domain and the previously known SS1 anti-mesothelin antibody (C), the disclosed MGHmesol L/H antibody (B-1) or the disclosed MGHmesol H/L antibody (B-2) specific lysis of different cancer cell lines (UTD control white). FIG. 11A compares SS1, MGHmesol L/H-BBz, and MGHmesol H/L-BBz specific lysis of BxPC-3 pancreatic cancer cells. FIG. 11B compares SS1, MGHmesol L/H-BBz, and MGHmesol H/L-BBz CAR T Cell specific lysis of Capan-2 cancer cells. FIG. 11C comparesSS1, MGHmesol L/H-BBz, and MGHmesol H/L-BBz specific lysis of NCI-H226 lung cancer cells.

FIGS. 12A-12B show schematics and transduction of the anti-mesothelin CAR TEAM constructs and control constructs. FIG. 12A shows schematics of the SS1 anti-mesothelin CAR TEAM FAP construct and 5 additional control constructs. FIG. 12B shows the transduction efficiency of anti-mesothelin SS1 CAR TEAM constructs and control constructs.

FIG. 13 shows results from a CART cell activation assay in plates with (dark grey) or without (light grey) mesothelin. CD69 expression of sorted CART cells (day 14) 6 hours after stimulation with mesothelin.

FIG. 14A-14C shows flow cytometric analysis (histograms) of supernatant from TEAM secreting CART cells with secondary His-tag labelling on target cell: FIG. 14A shows FAP-positive HFF cells and FIG. 14B shows CD19-positive K562 cells (representative example of 3 individual donors, day 20). FIG. 14C mean fluorescence intensity (MFI) ratio of 3 individual healthy donors. Statistical significance was calculated by the two tailed paired t-test.

FIG. 15A-15E shows an assay for determining the cytotoxic effects of secreted TEAMs. FIG. 15A shows a scheme of the co-culture assay. FIG. 15B, 15C, 15E show an impedance-based cytotoxicity assay of CART secreting TEAM against human foreskin fibroblasts in a 1:1 ratio (HFF in FIG. 15B-15C) and against cancer associated fibroblast in a 1:1 ratio (CAF-1 in FIG. 15E). FIG. 15D represents scheme of the transwell assay. FIG. 15A key: SS1 anti-mesothelin CAR FAP TEAM (1), supernatant from SS1 anti-mesothelin CAR FAP TEAM (2), SS1 anti-mesothelin CAR (3), and CD19 CAR (4), FIG. 15C key: SS1 anti-mesothelin CAR CD19 TEAM (1), SS1 anti-mesothelin CAR (2), and CD19 CAR (3). FIG. 15E key: SS1 anti-mesothelin CAR FAP TEAM (1), headless CAR FAP TEAM (2), anti-mesothelin CAR (3) CD19 CAR (4), CD19 CAR FAP TEAM (5), anti-mesothelin CAR CD19 TEAM (6) and UTD control (7).

FIG. 16 shows a timeline (top) and a graph (bottom) of an experiment evaluating the effect of anti-mesothelin CAR FAP TEAM on pancreatic tumor growth in mice. The top panel shows that pancreatic model cancer cells were injected into the mouse 3 days before treatment. The lower panel shows the tumor growth over time after treatment with anti-mesothelin CAR FAP TEAM (circle with horizonal lines), anti-mesothelin CAR CD19 TEAM (circle with zigzag lines), anti-CD19 CAR FAP TEAM (circle with vertical lines), or UTD (hollow circle). Tumor size is shown as mean values, error bars are standard deviation. Statistics were calculated with an unpaired t-test, *P=0.0192.

FIG. 17 shows graphs of avidity of the FAP TEAM molecule increases avidity between T cells and FAP expressing fibroblasts. HFF expressing FAP cells interacting with UTD (4, grey) and UTD CAR FAP TEAM (1) or anti-mesothelin CAR FAP TEAM (2) were analyzed using acoustic force microscopy. anti-CD19 CAR FAP TEAM (3) was a control. The % of bound cells was measured as a function of acoustic force (pN) (left panel). Fold increase of bound cells compared to UTD is shown in the right panel (paired t-test, *P<0.05).

### DETAILED DESCRIPTION

Provided herein are chimeric antigen receptors (CARs) and CAR T cells that target mesothelin. Without wishing to be bound by theory, MSLN (https://www.uniprot.org/uniprot/Q13421) is expressed on normal mesothelial cells in some tissues (e.g., pleura, pericardium, peritoneum) and in trace amounts in some epithelial cells (e.g., ovary, tunica vaginalis, rete testis, and fallopian tube), but is abundantly expressed in various cancer cells. See, e.g., Lv, Jiang, and Li, Peng. “Mesothelin as a biomarker for targeted therapy”. Biomark Res. 2019; 7:18; and Hassan et al. “Mesothelin Immunotherapy for Cancer: Ready for Prime Time?” J Clin Oncol. 2016 Dec. 1; 34 (34): 4171-4179, each of which is hereby incorporated by reference. Mesothelin may be used as a marker for cells associated with various cancers (e.g., over-expressed in various cancers), and CARs and CAR T cells that bind to mesothelin or a portion thereof may be used to treat subjects having, e.g., cancers associated with mesothelin expression.

### Chimeric Antigen Receptors (CARs)

The technology described herein provides improved chimeric antigen receptors (CARs) for use in immunotherapy. The following discusses CARs and the various improvements.

The terms “chimeric antigen receptor” or “CAR” or “CARs”, as used herein, refer to engineered T cell receptors, which graft a ligand or antigen specificity onto T cells (for example, naive T cells, central memory T cells, effector memory T cells or combinations thereof). CARs are also known as artificial T-cell receptors, chimeric T-cell receptors or chimeric immunoreceptors.

A CAR places a chimeric extracellular antigen-binding domain that specifically binds a target, e.g., a polypeptide, expressed on the surface of a cell to be targeted for a T cell response onto a construct including a transmembrane domain and intracellular domain(s) of a T cell receptor molecule. In some embodiments, the chimeric extracellular antigen-binding domain includes the antigen-binding domain(s) of an antibody reagent that specifically binds an antigen expressed on a cell to be targeted for a T cell response. In some embodiments, the chimeric extracellular antigen-binding domain includes a ligand that specifically binds an antigen expressed on a cell to be targeted for a T cell response.

As used herein, a “CART cell” or “CAR-T” refers to a T cell that expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

In some embodiments, the CAR polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity of a sequence selected from any one of SEQ ID NOs: 2-5. In some embodiments, the CAR polypeptide comprises an amino acid sequence of any on of SEQ ID NOs: 2-5. In some embodiments, the CAR polypeptide consists of an amino acid sequence of any one of SEQ ID NOs: 2-5. In some embodiments, the CAR excludes a CD8 signal peptide as described herein. As can be determined by those of skill in the art, various functionally similar or equivalent components of these CARs can be swapped or substituted with one another, as well as other similar or functionally equivalent components known in the art or listed herein.

### Extracellular Antigen-Binding Domain

As used herein, the term “extracellular antigen-binding domain” refers to a polypeptide found on the outside of the cell that is sufficient to facilitate binding to a target. The extracellular target binding domain will specifically bind to its binding partner, i.e., the target. As non-limiting examples, the extracellular antigen-binding domain can include an antigen-binding domain of an antibody or antibody reagent, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand: cognate partner binding can result in the alteration of the ligand-bearing receptor, or activate a physiological response, for example, the activation of a signaling pathway. In some embodiments, the ligand can be non-native to the genome. Optionally, the ligand has a conserved function across at least two species.

Any cell-surface moiety can be targeted by a CAR. Often, the target will be a cell-surface polypeptide that may be differentially or preferentially expressed on a cell that one wishes to target for a T cell response. To target Tregs, antibody reagents can be targeted against, e.g., Glycoprotein A Repetitions Predominant (GARP), latency-associated peptide (LAP), CD25, CTLA-4, ICOS, TNFR2, GITR, OX40, 4-1BB, and LAG-3. To target tumors or cancer cells, antibody domains can be targeted against, e.g., mesothelin, as described herein. Targeting tumor antigens that are specific to the tumors, e.g., mesothelin, can provide a means to target tumor cells while avoiding or at least limiting collateral damage to non-tumor cells or tissues.

In some embodiments, the target/antigen of the CAR is mesothelin.

### Hinge and Transmembrane Domains

Each CAR as described herein includes a transmembrane domain, e.g., a hinge/transmembrane domain, which joins the extracellular antigen-binding domain to the intracellular signaling domain. The binding domain of the CAR is optionally followed by one or more “hinge domains,” which plays a role in positioning the antigen binding domain away from the effector cell surface to enable proper cell/cell contact, antigen binding and activation. A CAR optionally includes one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8alpha), CD4, CD28, 4-1BB, and CD7, which may be wild-type hinge regions from these molecules or may be altered.

In some embodiments, the hinge region is derived from the hinge region of an immunoglobulin like protein (e.g., IgA, IgD, IgE, IgG, or IgM), CD28, or CD8. In some embodiments, the hinge domain includes a CD8a hinge region.

As used herein, “transmembrane domain” (TM domain) refers to the portion of the CAR that fuses the extracellular binding portion, optionally via a hinge domain, to the intracellular portion (e.g., the costimulatory domain and intracellular signaling domain) and anchors the CAR to the plasma membrane of the immune effector cell. The transmembrane domain is a generally hydrophobic region of the CAR, which crosses the plasma membrane of a cell. The TM domain can be the transmembrane region or fragment thereof a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR.

As used in relation to a transmembrane domain of a protein or polypeptide, “fragment thereof” refers to a portion of a transmembrane domain that is sufficient to anchor or attach a protein to a cell surface.

In some embodiments, the transmembrane domain or fragment thereof the CAR described herein includes a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGAI, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

As used herein, a “hinge/transmembrane domain” refers to a domain including both a hinge domain and a transmembrane domain. For example, a hinge/transmembrane domain can be derived from the hinge/transmembrane domain of CD8, CD28, CD7, or 4-1BB. In some embodiments, the hinge/transmembrane domain of a CAR or fragment thereof is derived from or includes the hinge/transmembrane domain of CD8 (e.g., any one of SEQ ID NOs: 24, or variants thereof). CD8 is an antigen preferentially found on the cell surface of cytotoxic T lymphocytes. CD8 mediates cell-cell interactions within the immune system, and acts as a T cell co-receptor. CD8 consists of an alpha (CD8alpha or CD8a) and beta (CD813 or CD8b) chain. CD8a sequences are known for a number of species, e.g., human CD8a, (NCBI Gene ID: 925) polypeptide (e.g., NCBI Ref Seq NP 001139345.1) and mRNA (e.g., NCBI Ref Seq NM 000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. In some embodiments, the hinge/transmembrane domain of a CAR or fragment thereof is derived from or includes the hinge/transmembrane domain of CD28 (e.g., comprising the amino acid sequence of SEQ ID NO: 38, or variants thereof, or as encoded by the nucleic acid sequence of SEQ ID NO: 39).

Homologs and/or orthologs of human CD8 are readily identified for such species by one of skill in the art, e.g., using the NCBI ortholog search function or searching available sequence data for a given species for sequence similar to a reference CD8 sequence.

In some embodiments, the CD8 hinge and transmembrane sequence corresponds to the amino acid sequence of SEQ ID NO: 24; or includes the sequence of SEQ ID NO: 24; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 24.

### Co-stimulatory Domains

Each CAR described herein optionally includes the intracellular domain of one or more co-stimulatory molecule or co-stimulatory domain. As used herein, the term “co-stimulatory domain” refers to an intracellular signaling domain of a co-stimulatory molecule. Co-stimulatory molecules are cell surface molecules other than antigen receptors or Fe receptors that provide a second signal required for efficient activation and function of T lymphocytes upon binding to antigen. The co-stimulatory domain can be, for example, the co-stimulatory domain of 4-1BB, CD27, CD28, or OX40. In one example, a 4-1BB intracellular domain (ICD) can be used (see, e.g., below and SEQ ID NOs: 25, or variants thereof). Additional illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some embodiments, the intracellular domain is the intracellular domain of 4-1 BB. 4-1 BB (CD137; TNFRS9) is an activation induced costimulatory molecule, and is an important regulator of immune responses.

4-1BB is a membrane receptor protein, also known as CD137, which is a member of the tumor necrosis factor (TNF) receptor superfamily. 4-1BB is expressed on activated T lymphocytes. 4-1BB sequences are known for a number of species, e.g., human 4-1 BB, also known as TNFRSF9 (NCBI Gene 25 ID: 3604) and mRNA (NCBI Reference Sequence: NM_001561.5). 4-1BB can refer to human 4-1BB, including naturally occurring variants, molecules, and alleles thereof. In some embodiments of any of the aspects, e.g., in veterinary applications, 4-1BB can refer to the 4-1BB of, e.g., dog, cat, cow, horse, pig, and the like. Homologs and/or orthologs of human 4-1BB are readily identified for such species by one of skill in the art, e.g., using the NCBI ortholog search function or searching available sequence data for a given species for sequence similar to a reference 4-1 BB sequence.

In some embodiments, the intracellular domain is the intracellular domain of a 4-1 BB. In some embodiments, the 4-1 BB intracellular domain corresponds to an amino acid sequence selected from SEQ ID NO: 25; or includes a sequence selected from SEQ ID NO: 25; or includes at least 75%, at least 80%, at least 85%, 35 at least 90%, at least 95%, at least 96%, at least 97%, at least 98%, at least 99%, or at least 100% sequence identity to a sequence selected from SEQ ID NO: 25.

### Intracellular Signaling Domains

The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3-zeta signals upon antigen binding. So-called “second-generation” CARs include those that provide both co-stimulation (e.g., CD28 or CD137) and activation (CD3-zeta) domains, and so-called “third-generation” CARs include those that provide multiple costimulatory (e.g., CD28 and CD137) domains and activation domains (e.g., CD3-zeta). In various embodiments, the CAR is selected to have high affinity or avidity for the target/antigen—for example, antibody-derived target or antigen binding domains will generally have higher affinity and/or avidity for the target antigen than would a naturally occurring T cell receptor. This property, combined with the high specificity one can select for an antibody provides highly specific T cell targeting by CART cells.

CARs as described herein include an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. In various examples, the intracellular signaling domain is from CD3-zeta; (see, e.g., below). Additional non-limiting examples of immunoreceptor tyrosine-based activation motif (ITAM)-containing intracellular signaling domains that are of particular use in the technology include those derived from TCR-zeta; FcR-gamma, FcR-beta, CD3-gamma, CD3-theta, CD3-sigma, CD3-eta, CD3-epsilon, CD3-zeta; CD22, CD79a, CD79b, and CD66d.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3-gamma chain, a CD3delta chain, and two CD3-epsilon chains.

These chains associate with a molecule known as the T cell receptor (TCR) and the CD3-zeta to generate an activation signal in T lymphocytes. A complete TCR complex includes a TCR, CD3-zeta; and the complete CD3 complex.

In some embodiments of any aspect, a CAR polypeptide described herein includes an intracellular signaling domain that includes an Immunoreceptor Tyrosine-based Activation Motif or ITAM from CD3-zeta, including variants of CD3-zeta; such as ITAM-mutated CD3-zeta, CD3-eta, or CD3-theta. In some embodiments of any aspect, the ITAM includes three motifs of ITAM of CD3-zeta; (ITAM3). In some embodiments of any aspect, the three motifs of ITAM of CD3-zeta; are not mutated and, therefore, include native or wild-type sequences. In some embodiments, the CD3-zeta; sequence includes the sequence of a CD3-zeta; as set forth in the sequences provided herein, e.g., a CD3-zeta; sequence of one of SEQ ID NOs: 26-27, or variants thereof.

For example, a CAR polypeptide described herein includes the intracellular signaling domain of CD3-zeta. In some embodiments, the CD3-zeta; intracellular signaling domain corresponds to an amino acid sequence selected from any one of SEQ ID NOs: 26-27; or includes a sequence selected from any one of SEQ ID NOs: 26-27; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from any one of SEQ ID NOs: 26-27.

Individual CAR and other construct components as described herein can be used with one another and swapped in and out of various constructs described herein, as can be determined by those of skill in the art. Each of these components can include or consist of any of the corresponding sequences set forth herein, or variants thereof.

A more detailed description of CARs and CART cells can be found in Maus et al., Blood 123:2624-2635, 2014; Reardon et al., Neuro-Oncology 16:1441-1458, 2014; Hoyos et al., Haematologica 97:1622, 2012; Byrd et al., J. Clin. Oncol. 32:3039-3047, 2014; Maher et al., Cancer Res 69:4559-4562, 2009; and Tamada et al., Clin. Cancer Res. 18:6436-6445, 2012; each of which is incorporated by reference herein in its entirety.

### Signal Peptide

In some embodiments, a CAR polypeptide as described herein includes a signal peptide. Signal peptides can be derived from any protein that has an extracellular domain or is secreted. A CAR polypeptide as described herein may include any signal peptides known in the art. In some embodiments, the CAR polypeptide includes a CD8 signal peptide, e.g., a CD8 signal peptide corresponding to the amino acid sequence of SEQ ID NO: 29, or including the amino acid sequence of SEQ ID NO: 29, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 1 00% sequence identity to the sequence of SEQ ID NO: 29.

In further embodiments, a CAR polypeptide described herein may optionally exclude one of the signal peptides described herein, e.g., a CD8 signal peptide of SEQ ID NO: 29 or an IgK signal peptide of SEQ ID NO: 30.

### Linker Domain

In some embodiments, the CAR further includes a linker domain. As used herein, “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Linker sequences useful for the CARs of the disclosure can be from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the CARs of the disclosure include, but are not limited to, glycine/serine linkers, e.g., GGGSGGGSGGGS (SEQ ID NO: 31) and Gly4Ser (G4S) (SEQ ID NO: 48) linkers such as (G4S) 3 (GGGGSGGGGSGGGGS (SEQ ID NO: 32)) and (G4S) 4 (GGGGSGGGGSGGGGSGGGGS (SEQ ID NO: 33)); the linker sequence of GSTSGSGKPGSGEGSTKG (SEQ ID NO: 34) as described by Whitlow et al., Protein Eng. 6 (8): 989-95, 1993, the contents of which are incorporated herein by reference in its entirety; the linker sequence of GGSSRSSSSGGGGSGGGG (SEQ ID NO: 35) as described by Andris-Widhopf et al., Cold Spring Harb. Protoc. 2011 (9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18 (1): 75-80, 2000, the contents of which are incorporated herein by reference in its entirety. Longer linkers may be used when it is desirable to ensure that two adjacent domains do not sterically interfere with one another.

Furthermore, linkers may be cleavable or non-cleavable. Examples of cleavable linkers include 2A linkers (e.g., P2A and T2A (SEQ ID NO: 37), 2A-like linkers or functional equivalents thereof and combinations thereof.

For example, a P2A linker sequence can correspond to the amino acid sequence of SEQ ID NO: 38. In various examples, linkers having sequences as set forth herein, or variants thereof, are used. It is to be understood that the indication of a particular linker in a construct in a particular location does not mean that only that linker can be used there. Rather, different linker sequences (e.g., P2A and T2A) can be swapped with one another (e.g., in the context of the constructs of the present disclosure), as can be determined by those of skill in the art. In some embodiments, the linker region is T2A derived from Thosea asigna virus. Non-limiting examples of linkers that can be used in this technology include T2A, P2A, E2A, BmCPV2A, and BmlFV2A. Linkers such as these can be used in the context of polyproteins, such as those described below. For example, they can be used to separate a CAR component of a polyprotein from a therapeutic agent (e.g., an antibody, such as a scFv, single domain antibody (e.g., a camelid antibody), or a bispecific antibody (e.g., a TEAM)) component of a polyprotein (see below).

### Reporter Molecule

In some embodiments, a CAR as described herein optionally further includes a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CART cell, the first CAR and the second CAR can express different or the same reporter molecule. In another embodiment, a CAR as described herein further includes a reporter molecule (for example hygromycin phosphotransferase (hph)) that can be imaged alone or in combination with a substrate or chemical (for example 9-[4-[18F] fluoro-3-(hydroxymethyl)butyl] guanine ([18F]FHBG)). In another embodiment, a CAR as described herein further includes nanoparticles at can be readily imaged using non-invasive techniques (e.g., gold nanoparticles (GNP) functionalized with 64Cu2+). Labeling of CART cells for non-invasive imaging is reviewed, for example in Bhatnagar et al., Integr. Biol. (Camb). 5 (1): 231-238, 2013, and Keu et al., Sci. Transl. Med. 18; 9 (373), 2017, which are incorporated herein by reference in their entireties.

In some embodiments, GFP and mCherry may be used as fluorescent tags for imaging a CAR expressed on a T cell (e.g., a CART cell). It is expected that essentially any fluorescent protein known in the art can be used as a fluorescent tag for this purpose. For clinical applications, the CAR need not include a fluorescent tag or fluorescent protein. In each instance of particular constructs provided herein, therefore, any markers present in the constructs can be removed. The disclosure includes the constructs with or without the markers. Accordingly, when a specific construct is referenced herein, it can be considered with or without any markers or tags (including, e.g., histidine tags, such as the histidine tag of HHHHHH (SEQ ID NO: 39)) as being included within the disclosure.

### Antibody Reagents

In various embodiments, the CARs described herein comprise an antibody reagent or an antigen-binding domain thereof as an extracellular target-binding domain.

As used herein, the term “antibody reagent” refers to a polypeptide that includes at least one immunoglobulin variable domain or immunoglobulin variable domain sequence and which specifically binds a given antigen. In some embodiments, an antibody reagent can include an antibody or a polypeptide including an antigen-binding domain of an antibody. In some embodiments of any of the aspects, an antibody reagent can include a monoclonal antibody or a polypeptide including an antigen-binding domain of a monoclonal antibody. For example, an antibody can include a heavy (H) chain variable region (abbreviated herein as VH), and a light (L) chain variable region (abbreviated herein as VL). In some embodiments, an antibody includes two heavy (H) chain variable regions and two light (L) chain variable regions. In some embodiments, the antibody reagent is a bispecific antibody reagent.

The term “antibody reagent” encompasses antigen-binding fragments of antibodies (e.g., single chain antibodies, Fab and sFab fragments, F (ab′) 2, Fd fragments, Fv fragments, scFv, CDRs, and domain antibody (dAb) fragments (see, e.g., de Wildt et al., Eur. J. Immunol. 26 (3): 629-639, 1996; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. In some embodiments, the CAR comprises an antibody reagent. In some embodiments, the therapeutic agent comprises an antibody reagent.

Fully human antibody binding domains can be selected, for example, from phage display libraries using methods known to those of ordinary skill in the art. Furthermore, antibody reagents include single domain antibodies, such as camelid antibodies.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A. et al. (1991) Sequences of Proteins of Immunological Interest, Fifth Edition, U.S. Department of Health and Human Services, NIH Publication No. 91-3242, and Chothia et al., J. Mol. Biol. 196:901-917, 1987; each of which is incorporated by reference herein in its entirety). Each VH and VL is typically composed of three CDRs and four FRs, arranged from amino-terminus to carboxy-terminus in the following order: FR1, CDR1, FR2, CDR2, FR3, CDR3, FR4.

In some embodiments, the antibody or antibody reagent is not a human antibody or antibody reagent (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In some embodiments, the extracellular target binding domain of a CAR includes or consists essentially of a single-chain Fv (scFv) fragment created by fusing the VH and VL domains of an antibody, generally a monoclonal antibody, via a flexible linker peptide. In various embodiments, the scFv is fused to a transmembrane domain and to a T cell receptor intracellular signaling domain, e.g., an engineered intracellular signaling domain as described herein. In another embodiment, the extracellular target binding domain of a CAR includes a camelid antibody.

In some embodiments, a CAR, antibody, or antigen-binding domain binds to mesothelin. #Mesothelin sequences are known for a number of species, e.g., human mesothelin (NCBI Gene ID: 10232) and polypeptide (e.g., pre-protein NCBI Ref Seq: NP_005814.2). Mesothelin can refer to human mesothelin, including naturally occurring variants, molecules, and alleles thereof. Homologs and/or orthologs of human mesothelin are readily identified for such species by one of skill in the art, e.g., using the mesothelin ortholog search function or searching available sequence data for a given species for sequence similar to a reference mesothelin sequence. In some embodiments, a CAR, antibody, or antigen-binding domain that specifically binds mesothelin specifically binds a homolog and/or ortholog of human mesothelin, e.g., in addition to human mesothelin.

The terms “anti-mesothelin antibody,” “an antibody that binds to mesothelin,” and “an antibody that specifically binds to mesothelin” refer to an antibody reagent that is capable of binding mesothelin with sufficient affinity such that the antibody is useful as a preventative, diagnostic, and/or therapeutic agent in targeting mesothelin. In one embodiment, the extent of binding of an anti-mesothelin antibody to an unrelated, non-mesothelin protein is less than about 10% of the binding of the antibody to mesothelin as measured, e.g., by a radioimmunoassay (RIA). In certain embodiments, an antibody that binds to mesothelin has a dissociation constant (K_{D}) of ≤1μ, ≤100 nM, ≤10 nM, ≤1 nM, or ≤0.1 nM, (e.g. 10⁻⁶, 10⁻⁷, or 10⁻⁸ M or less, e.g. from 10⁻⁶ M to 10⁻⁹ M). In certain embodiments, an antibody that binds to mesothelin has a dissociation constant (K_{D}) of ≤1 nM, ≤0.1 nM, ≤0.01 nM, or ≤0.001 nM, (e.g. 10⁻⁹, 10⁻¹⁰, 10⁻¹¹, or 10⁻¹² M or less, e.g. from 10⁻⁹ M to 10⁻¹² M).

In some embodiments, the anti-mesothelin antibody reagent is MGHmeso1. In some embodiments, the anti-mesothelin antibody reagent comprises the complementarity determining regions (CDRs) of SEQ ID NOs: 13-18, or comprises CDR sequences with no more than 1, 2, or 3 amino acid substitutions to a given CDR of SEQ ID NOs: 13-18. In some embodiments, the anti-mesothelin antibody reagent comprises the variable heavy (VH) and/or variable light (VL) of SEQ ID NOs: 19-20, or includes VH and/or VL sequences with at least 75%, at least 80%, at least 85%, at least 90%, at least 95%, at least 96%, at least 97%, at least 98%, at least 99%, or greater sequence identity to the sequences of SEQ ID NOs: 19-20. The VH may be positioned N-terminal to the VL, or the VL may be positioned N-terminal to the VH.

In some embodiments, the antibody reagent comprises daclizumab or an antigen-binding fragment thereof. Antibody reagents can also be targeted against any other antigens described herein or known in the art. In addition to optionally delivering antibody reagents, as described herein, the CART cells of the disclosure can be used to deliver other therapeutic agents including, but not limited to, cytokines and toxins. In some embodiments, the therapeutic reagent comprises an antibody reagent that binds to any one of epidermal growth factor receptor variant III (EGFRvIII), epidermal growth factor receptor (EGFR), CD19, prostate-specific membrane antigen (PSMA), or IL-13 receptor alpha 2 (IL-13Ra2). In some embodiments, the therapeutic reagent comprises an antibody reagent that binds to two or more of epidermal growth factor receptor variant III (EGFRvIII), epidermal growth factor receptor (EGFR), CD19, prostate-specific membrane antigen (PSMA), or IL-13 receptor alpha 2 (IL-13Ra2).

In some embodiments, the antibody reagent binds to a tumor associated-antigen. Non-limiting examples of additional tumor antigens or other antigen of interest include activated fibroblast marker, CD19, CD37, BCMA (tumor necrosis factor receptor superfamily member 17 (TNFRSF17); NCBI Gene ID: 608; NCBI Ref Seq: NP 001183.2 and mRNA (e.g., NCBI Ref Seq: NM_001192.2)), CEA, immature laminin receptor, TAG-72, HPV E6 and E7, BING-4, calcium-activated chloride channel 2, cyclin B1, 9D7, Ep-CAM, EphA3, 15 her2/neu, telomerase, EGFR, EGFRviii SAP-1, survivin, BAGE family, CAGE family, GAGE family, MAGE family, SAGE family, XAGE family, NY-ESO-1/LAGE-1, PRAME, SSX-2, Melan-NMART-1, gp100/pmel 17, tyrosinase, TRP-1/-2, MCIR, BRCA1/2, CDK4, MART-2, p53, Ras, MUCI, TGF-BetaRII, IL-15, IL-13Ra2, and CSF1 R. In some embodiments, the activated fibroblast marker comprises any one of aSMA (ACTA2), fibroblast activation protein (FAP), platelet derived growth factor receptor-α and -β (PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1).

### Multispecific Antibody Reagents

In some embodiments, an antibody reagent is multispecific, capable of specifically binding two or more target antigens. In some embodiments, an antibody reagent is bispecific (i.e., a bispecific antibody reagent). In some embodiments, a bispecific antibody reagent specifically binds a first antigen (e.g., mesothelin) and a second antigen. In some embodiments, the second antigen is a tumor antigen. In some embodiments, the second antigen is expressed by (e.g., on the surface of) mesothelin-expressing cancer cells. In some embodiments, the second antigen is epidermal growth factor receptor (EGFR), BCMA, CD19, CD37, carcinoembryonic antigen (CEA), epithelial cell adhesion molecule (EpCAM), EphA3, Her2/neu, MUCI, TGF-βRII, colony stimulating factor 1 receptor (CSFIR), prostate-specific membrane antigen (PSMA), prostate stem cell antigen (PSCA), or any other tumor antigen described herein. In some embodiments, a multispecific antibody reagent is a TEAM, as described herein.

A multispecific antibody reagent may comprise a first binding domain comprising a full-length antibody or antibody fragment thereof that binds to a first antigen (e.g., mesothelin) and a second binding domain comprising a different full-length antibody or antibody fragment thereof that binds to a second antigen (e.g., a tumor antigen). The first and second binding domains may be connected by a linker sequence, e.g., a linker sequence described herein.

### Therapeutic Agents Delivered by CAR T Cells

As noted above, the CART cells of the disclosure can optionally be used to deliver therapeutic agents, e.g., antibody reagents or other therapeutic molecules, such as cytokines, to tumors (i.e., to the tumor microenvironment). In some embodiments, the cytokine is an interferon, interleukin or growth factor. In some embodiments, the therapeutic agent is encoded by the same nucleic acid molecule as the CAR, thus facilitating transduction of cells (e.g., T cells) to express both a CAR and a therapeutic agent, e.g., an antibody reagent or cytokine. In such examples, the therapeutic agent (e.g., an antibody reagent or cytokine) can be expressed, e.g., such that it is separated from the CAR (and optionally other proteins, e.g., markers) by cleavable linker sequences (e.g., a 2A ribosomal skip sequence, and internal ribosome entry site (IRS), or a 2A linker (2A peptide), such as, e.g., P2A or T2A; see above). In some embodiments, the therapeutic agent is encoded on a different nucleic acid molecule than the CAR.

In some embodiments, the therapeutic agent (e.g., an antibody reagent or cytokine) can be expressed under the control of the same promoter as the CAR (e.g., by an EF1-alpha a promoter), and can be constitutively expressed. In some embodiments, the therapeutic agent (e.g., an antibody reagent or cytokine) is expressed under the control of an inducible promoter, e.g., a promoter that is expressed upon T cell activation (e.g., an NFAT promoter). Such an inducible promoter can be used, e.g., to ensure that the antibody is expressed only upon T cell activation, and thus only, e.g., when the CART cell is within the tumor microenvironment, to which locale it may be advantageous to have antibody production limited. As is understood in the art, the CAR coding sequences can be 5′ or 3′ to the therapeutic agent (e.g., an antibody reagent or cytokine) coding sequences in various vector designs within the disclosure.

In some embodiments, the therapeutic agent includes an IgK signal peptide, e.g., an IgK signal peptide corresponding to the amino acid sequence of SEQ ID NO: 30, or including the amino acid sequence of SEQ ID NO: 30, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 1 00% sequence identity to the sequence of SEQ ID NO: 30.

In various examples, the therapeutic agent is an antibody reagent. The antibody reagent expressed within a CART cell (e.g., from the same nucleic acid molecule as the CAR) can be a single chain antibody (e.g., an scFv) or a single domain antibody (e.g., a camelid) as described herein. In the case of single chain antibodies, the light (L) and heavy (H) chains may be in the order (N-terminal to C-terminal) L-H or H-L, and optionally may be separated from one another by a linker (e.g., a glycine-based linker). In further examples, the antibody reagent is a bispecific antibody including, e.g., T cell engaging molecules (TEAMs), described below.

### T Cell Engaging Molecules (TEAMs)

In some embodiments, the therapeutic agent delivered by a CART cell as described herein is a T cell engaging molecule (TEAM) #(also referred to in the literature as bispecific T cell engagers or BiTES™). By “T cell engaging molecules,” “TEAM antibody constructs,” or TEAMs” is meant polypeptides that each include tandemly linked single-chain variable fragments (scFvs). Optionally, the scFvs are linked by a linker (e.g., a glycine-rich linker). One scFv of the TEAM binds to the T cell receptor (TCR) (e.g., to the CD3£ subunit) and the other binds to a target antigen (e.g., a tumor antigen). Such molecules can target T cells by binding to a T cell antigen (e.g., by binding CD3) as well as a target antigen, e.g., a tumor antigen. Exemplary tumor antigens include mesothelin (also see below). The TEAMs can be used to augment the T cell response in, e.g., the tumor microenvironment. The two components of a TEAM can optionally be separated from one another by a inker as described herein (e.g., a glycine-based linker), and may also be connected in either orientation, e.g., with the anti-CD3 component N-terminal to the anti-target antigen component, or vice versa. The anti-CD3 component or the anti-target antigen component of the TEAM may include any of the antibody reagents described herein.

The CART cell secreted TEAMs may, e.g., stimulate the CART cell itself, or operate in a paracrine fashion by redirecting nonspecific bystander T cells against tumors and therefore enhance the anti-tumor effects of CART cell immunotherapy. CART cell-mediated TEAM secretion may allow for the reduction of risk of undesired TEAM activity in systemic tissues by directing TEAM secretion to the tumor microenvironment. Exemplary TEAM constructs are provided below; however, TEAMs other than those described herein may also be useful for the CAR T cells and methods of the disclosure.

An exemplary TEAM is an anti-CD19 TEAM including an anti-CD19 scFv and an anti-CD3 scFv (also referred to herein as TEAM-CD19). The anti-CD19 scFv may be arranged in the VH-VL orientation, or in the VL-VH orientation. In certain embodiments, the anti-CD19 scFv corresponds to the amino acid sequence of SEQ ID NO: 37, or includes the amino acid sequence of SEQ ID NO: 37, or includes an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 37.

In some embodiments, the anti-CD3 scFv of any of the TEAMs described herein may be arranged in the VH-VL orientation, or in the VL-VH orientation. In some embodiments, the anti-CD3 VH comprises the amino acid sequence of SEQ ID NO: 9 or an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 9. In some embodiments, the anti-CD3 VL comprises the amino acid sequence of SEQ ID NO: 10 or an amino acid sequence having at least 75%, at least 80%, at least 85%, at least 90%, at least 95%, at least 96%, at least 97%, at least 98%, at least 99%, or greater sequence identity to the amino acid sequence of SEQ ID NO: 10.

In some embodiments, the TEAM comprises an antibody that binds to glycoprotein A repetitions predominant (GARP), latency-associated peptide (LAP), CD25, cytotoxic T lymphocyte-associated antigen-4 (CTLA-4). In some embodiments, the TEAM comprises an amino acid sequence of any one of SEQ ID NOs: 11-12 or an amino acid sequence having at least 75%, at least 80%, at least 85%, at least 90%, at least 95%, at least 96%, at least 97%, at least 98%, at least 99%, or greater sequence identity to the amino acid sequence of any one of SEQ ID NOs: 11-12.

In some embodiments, the TEAM comprises an antibody reagent that binds to fibroblast activation protein (FAP).

In some embodiments, the anti-FAP antibody reagent is Sibrotuzumab. In some embodiments, the anti-FAP antibody reagent comprises the variable heavy (VH) of SEQ ID NO: 6 or comprises VH sequences with at least 75%, at least 80%, at least 85%, at least 90%, at least 95%, at least 96%, at least 97%, at least 98%, at least 99%, or greater sequence identity to SEQ ID NOs: 6. In some embodiments, the anti-FAP antibody reagent comprises the variable light (VL) of SEQ ID NO: 7 or includes VL sequences with at least 75%, at least 80%, at least 85%, at least 90%, at least 95%, at least 96%, at least 97%, at least 98%, at least 99%, or greater sequence identity to the sequences of SEQ ID NOs: 7. The VH may be positioned N-terminal to the VL, or the VL may be positioned N-terminal to the VH. In some embodiments, the anti-FAP antibody reagent comprises SEQ ID NO: 8 or comprises a sequence with at least 75%, at least 80%, at least 85%, at least 90%, at least 95%, at least 96%, at least 97%, at least 98%, at least 99%, or greater sequence identity to SEQ ID NO: 8.

In some embodiments, the TEAM comprises an antibody reagent that binds to fibroblast activation protein (FAP) and an antibody reagent that binds to CD3. In some embodiments, the FAP antibody reagent is encoded upstream of the CD3 scFv.

In some embodiments, the CAR and TEAM are encoded on the same polypeptide. In some embodiments, the CAR and TEAM are encoded on the same polypeptide are separated by a linker domain as described above. In some embodiments, the linker domain in cleavable. In some embodiments, the CAR is an anti-mesothelin CAR and the TEAM is a fibroblast activation protein (FAP) and CD3 TEAM. In some embodiments, the CAR is an anti-mesothelin CAR and the TEAM comprises an anti-FAP scFv and an anti-CD3 scFv. In some embodiments, the CAR and TEAM polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity of SEQ ID NO: 1. In some embodiments, the CAR and team polypeptide comprises an amino acid sequence SEQ ID NO: 1. In some embodiments, the CAR and team polypeptide consists of an amino acid sequence SEQ ID NO: 1.

In some embodiments, the TEAM enhances binding of an immune cell to a cancer compared to binding of the immune cell to the cancer in the absence of the TEAM. In some embodiments, the TEAM enhances binding of an immune cell to a cancer compared to binding of the immune cell to the cancer without a TEAM and without a CAR that binds to an antigen on the immune cell. In some embodiments, the TEAM increases binding of the immune cell to the cancer by at least 10% (e.g., at least 10%, at least 20%, at least 30%, at least 40%, at least 50%, at least 60%, at least 70%, at least 80%, at least 90%, at least 100%, at least 125%, at least 150%, at least 175%, at least 200%, or at least 250%). In some embodiments, the TEAM increases immune cell binding to the cancer by 10%-20%, 10%-30%, 10%-50%, 10%-100%, 10%-150%, 10%-200%, 10%-250%, 50%-100%, 50%-150%, 50%-200%, 50%-250%, 100%-150%, 100%-200%, 100%-250%, 20%-30%, 20%-40%, 20%-50%, 20%-60%, 20%-70%, 30%-40%, 30%-50%, 30%-60%, or 30%-70%. In some embodiments, the TEAM increases immune cell binding to the cancer by 20%-40%. In some embodiments, the TEAM increases immune cell binding to the cancer by 100%-250%. In some embodiments, the immune cell is a T cell. In some embodiments, the immune cell is a CAR T cell. In some embodiments, the TEAM is a FAP TEAM.

### CAR T Cells

One aspect of the technology described herein relates to a mammalian cell including any of the CAR polypeptides described herein (optionally together with another therapeutic agent (e.g., an antibody reagent (e.g., a scFv, a camelid antibody, or a TEAM) or a cytokine)); or a nucleic acid encoding any of the CAR polypeptides described herein (optionally together with another therapeutic agent (e.g., an antibody reagent (e.g., a scFv, a camelid antibody, or a TEAM) or a cytokine)). In some embodiments, the mammalian cell includes an antibody, antibody reagent, antigen-binding portion thereof, any of the CARs described herein, or a cytokine, or a nucleic acid encoding such an antibody, antibody reagent, antigen-binding portion thereof, any of the CARs described herein, or a cytokine. The mammalian cell or tissue can be of human, primate, hamster, rabbit, rodent, cow, pig, sheep, horse, goat, dog or cat origin, but any other mammalian cell may be used. In a preferred embodiment of any aspect, the mammalian cell is human.

In some embodiments of any aspect, the mammalian cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the immune cell is a T cell; a NK cell; a NKT cell; lymphocytes, such as B cells and T cells; and myeloid cells, such as monocytes, macrophages, eosinophils, mast cells, basophils, and granulocytes. In some embodiments, the immune cell is a T cell.

In some embodiments, the immune cell is obtained from an individual having or diagnosed as having cancer, a plasma cell disorder, or autoimmune disease.

In some embodiments, a mammalian cell, e.g., a T cell, can be engineered to include any of the CAR polypeptides described herein and a therapeutic agent, as described herein; or a nucleic acid encoding any of the CAR polypeptides and a therapeutic agent described herein. T cells can be obtained from a subject using standard techniques known in the field. For example, T cells can be isolated from peripheral blood taken from a donor or patient. T cells can be isolated from a mammal. Preferably, T cells are isolated from a human.

In some embodiments, the CAR T cell comprises an anti-mesothelin CAR as described herein. In some embodiments, the CAR T cell comprises a nucleic acid sequence encoding a CAR of any one of SEQ ID NOs: 2-5 or a CAR-TEAM polypeptide of SEQ ID NO: 1.

### CAR T and Therapeutic Agent Nucleic Acids Sequences

Also provided are nucleic acid constructs and vectors encoding (i) a CAR polypeptide (e.g., of any one of SEQ ID NOs: SEQ ID NO: 2-5) or (ii) a polyprotein including a CAR polypeptide and a therapeutic agent (e.g., of SEQ ID NO: 1) described herein for use in generating CART cells.

In some embodiments, the disclosure provides constructs that each include separate coding sequences for multiple proteins to be expressed in a CART cell of the disclosure. These separate coding sequences can be separated from one another by a cleavable linker sequence as described herein. For example, sequences encoding viral 2A proteins (e.g., T2A and P2A) can be placed between the separate genes and, when transcribed, can direct cleavage of the generated polyprotein. As noted above, constructs and vectors of the disclosure can include any of a number of different combinations of sequences. For example, a construct or vector of the disclosure can include sequences encoding one a CAR as described herein, optionally in combination with a therapeutic agent (e.g., an antibody reagent (e.g., a single chain antibody, a single domain antibody (e.g., a camelid), or a bispecific antibody (e.g., a TEAM)) or a cytokine) as described herein.

Efficient expression of proteins in CART cells as described herein can be assessed using standard assays that detect the mRNA, DNA, or gene product of the nucleic acid encoding the proteins. For example, RT-PCR, FAGS, northern blotting, western blotting, ELISA, or immunohistochemistry can be used. The proteins described herein can be constitutively expressed or inducibly expressed. In some embodiments, the proteins are encoded by a recombinant nucleic acid sequence. For example, the disclosure provides a vector that includes a first polynucleotide sequence encoding a CAR, wherein the CAR includes an extracellular domain including an antigen-binding sequence that binds to, e.g., a tumor antigen or a Treg-associated antigen, and, optionally, a second polynucleotide sequence encoding a therapeutic agent (e.g., an antibody reagent (e.g., a single chain antibody, a single domain antibody (e.g., a camelid), or a bispecific antibody (e.g., a TEAM)) or a cytokine).

In some embodiments, the first polynucleotide sequence and the second polynucleotide sequence are each operably linked to a promoter. In some embodiments, the first polynucleotide sequence is operably linked to a first promoter and the second polynucleotide sequence is operably linked to a second promoter. The promoter can be a constitutively expressed promoter (e.g., an EFla promoter) or an inducibly expressed promoter (e.g., a NFAT promoter).

In some embodiments, expression of the CAR and therapeutic agent are driven by the same promoter, e.g., a constitutively expressed promoter (e.g., an EF1 a promoter). In other embodiments, expression of the CAR and therapeutic agent are driven by different promoters. For instance, expression of the CAR can be driven by a constitutively expressed promoter (e.g., an EF1 a promoter) while expression of the therapeutic agent can be driven by an inducibly expressed promoter (e.g., a NFAT promoter). The polynucleotide sequence encoding the CAR can be located upstream of the polynucleotide sequence encoding the therapeutic agent, or the polynucleotide sequence encoding the therapeutic agent can be located upstream the polynucleotide sequence encoding the CAR.

In some embodiments, the polynucleotides can include the expression of a suicide gene. This can be done to facilitate external, drug-mediated control of administered cells. For example, by use of a suicide gene, modified cells can be depleted from the patient in case of, e.g., an adverse event. In some embodiments, the FK506 binding domain is fused to the caspase9 pro—apoptotic molecule. T cells engineered in this manner are rendered sensitive to the immunosuppressive drug tacrolimus. Other examples of suicide genes are thymidine kinase (TK), CD20, thymidylate kinase, truncated prostate-specific membrane antigen (PSMA), truncated low affinity nerve growth factor receptor (LNGFR), truncated co· 19, and modified Fas, which can be triggered for conditional ablation by the administration of specific molecules (e.g., ganciclovir to TK+ceils) or antibodies or antibody-drug conjugates.

Constructs including sequences encoding proteins for expression in the CAR T cells can be included within vectors. In various examples, the vectors are retroviral vectors. Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Galster Academic Press (ISBN: 978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, MD. In various embodiments, the protein is expressed in the T cell by transfection or electroporation of an expression vector including nucleic acid encoding the protein using vectors and methods that are known in the art. In some embodiments, the vector is a viral vector or a non-viral vector.

In some embodiments, the viral vector is a retroviral vector (e.g., a lentiviral vector), an adenovirus vector, or an adeno-associated virus vector.

In some embodiment a composition that includes a vector includes a first polynucleotide sequence encoding a CAR, wherein the CAR includes an extracellular domain including a sequence that specifically binds to a tumor antigen or a Treg-associated antigen, and, optionally, a second polynucleotide sequence encoding a therapeutic agent. In certain embodiments, when the therapeutic agent is an antibody reagent (e.g., a single chain antibody, a single domain antibody (e.g., a camelid), or a bispecific antibody (e.g., a TEAM)), the antibody reagent specifically binds to a tumor antigen or a Treg-associated antigen.

### CAR and Therapeutic Agent Expression

In some embodiments of any aspect, any of the CAR polypeptides (optionally together with an antibody reagent as described herein or a cytokine) described herein are expressed from a lentiviral vector. The lentiviral vector is used to express the CAR polypeptide (and optionally also the antibody reagent or cytokine) in a cell using infection standard techniques.

Retroviruses, such as lentiviruses, provide a convenient platform for delivery of nucleic acid sequences encoding a gene or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Galster Academic Press (ISBN: 978-1-90455-55-4); and Hu et al., Pharmacological Reviews 52:493-512, 2000; which are each incorporated by reference herein in their entirety. Lentiviral system for efficient DNA delivery can be purchased from OriGene; Rockville, MD. In some embodiments, the CAR polypeptide (and optionally the antibody reagent or cytokine) of any of the CARs described herein is expressed in a mammalian cell via transfection or electroporation of an expression vector including a nucleic acid encoding the CAR.

Transfection or electroporation methods are known in the art.

Efficient expression of the CAR polypeptide (and optionally the antibody reagent or cytokine) of any of the polypeptides described herein can be assessed using standard assays that detect the mRNA, DNA, or gene product of the nucleic acid encoding the CAR (and optional antibody reagent or cytokine), such as RT-PCR, FAGS, northern blotting, western blotting, ELISA, or immunohistochemistry.

In some embodiments, the CAR polypeptide (and optional antibody reagent or cytokine) described herein is constitutively expressed. In other embodiments, the CAR polypeptide is constitutively expressed and the optional antibody reagent or cytokine is inducibly expressed. In some embodiments, the CAR polypeptide (and optional antibody reagent or cytokine) described herein is encoded by recombinant nucleic acid sequence.

### Sequences

TABLE 1
CAR components and TEAM sequences.

| SEQ | | |
|---|---|---|
| ID | | |
| NO: | Description | Sequence |
| 1 | anti- | MALPVTALLLPLALLLHAARPQVQLQQSGPELEKPGASVKISCK |
| | mesothelin | ASGYSFTGYTMNWVKQSHGKSLEWIGLITPYNGASSYNQKFRG |
| | SS1 CART | KATLTVDKSSSTAYMDLLSLTSEDSAVYFCARGGYDGRGFDY |
| | FAP TEAM | WGQGTTVTVSSGGGGSGGGGSGGGGSDIELTQSPAIMSASPGE |
| | | KVTMTCSASSSVSYMHWYQQKSGTSPKRWIYDTSKLASGVPGR |
| | | FSGSGSGNSYSLTISSVEAEDDATYYCQQWSGYPLTFGAGTKLE |
| | | ITTTPAPRPPTPAPTIASQPLSLRPEACRPAAGGAVHTRGLDFAC |
| | | DIYIWAPLAGTCGVLLLSLVITLYCKRGRKKLLYIFKQPFMRPV |
| | | QTTQEEDGCSCRFPEEEEGGCELRVKFSRSADAPAYQQGQNQL |
| | | YNELNLGRREEYDVLDKRRGRDPEMGGKPRRKNPQEGLYNEL |
| | | QKDKMAEAYSEIGMKGERRRGKGHDGLYQGLSTATKDTYDAL |
| | | HMQALPPRSGGSGATNFSLLKQAGDVEENPGPMHMETDTLLL |
| | | WVLLLWVPGSTGDDIVMTQSPDSLAVSLGERATINCKSSQSLLY |
| | | SRNQKNYLAWYQQKPGQPPKLLIFWASTRESGVPDRFSGSGFG |
| | | TDFTLTISSLQAEDVAVYYCQQYFSYPLTFGQGTKVEIKGGGGS |
| | | GGGGSGGGGSQVQLVQSGAEVKKPGASVKVSCKTSRYTFTEYT |
| | | IHWVRQAPGQRLEWIGGINPNNGIPNYNQKFKGRVTITVDTSAS |
| | | TAYMELSSLRSEDTAVYYCARRRIAYGYDEGHAMDYWGQGTL |
| | | VTVSSGGGSDIKLQQSGAELARPGASVKMSCKTSGYTFTRYTM |
| | | HWVKQRPGQGLEWIGYINPSRGYTNYNQKFKDKATLTTDKSSS |
| | | TAYMQLSSLTSEDSAVYYCARYYDDHYCLDYWGQGTTLTVSS |
| | | VEGGSGGSGGSGGSGGVDDIQLTQSPAIMSASPGEKVTMTCRAS |
| | | SSVSYMNWYQQKSGTSPKRWIYDTSKVASGVPYRFSGSGSGTS |
| | | YSLTISSMEAEDAATYYCQQWSSNPLTFGAGTKLELKHHHHHH |
| | | SGGGGEGRGSLLTCGDVEENPGPRMVSKGEEDNMAIIKEFMRF |
| | | KVHMEGSVNGHEFEIEGEGEGRPYEGTQTAKLKVTKGGPLPFA |
| | | WDILSPQFMYGSKAYVKHPADIPDYLKLSFPEGFKWERVMNFE |
| | | DGGVVTVTQDSSLQDGEFIYKVKLRGTNFPSDGPVMQKKTMG |
| | | WEASSERMYPEDGALKGEIKQRLKLKDGGHYDAEVKTTYKAK |
| | | KPVQLPGAYNVNIKLDITSHNEDYTIVEQYERAEGRHSTGGMDE |
| | | YK |
| 40 | SS1 | QVQLQQSGPELEKPGASVKISCKASGYSFTGYTMNWVKQSHGK |
| | antibody | SLEWIGLITPYNGASSYNQKFRGKATLTVDKSSSTAYMDLLSLT |
| | VH | SEDSAVYFCARGGYDGRGFDYWGQGTTVTVSS |
| 41 | SS1 antibody VL | 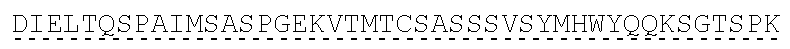 |
| | | 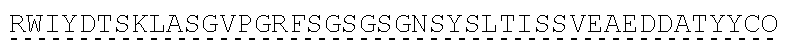 |
| | | 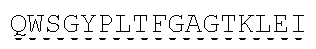 |
| 42 | SS1 | GYSFTGYTMN |
| | antibody | |
| | CDR-H1 | |
| 43 | SS1 | LITPYNGASSYNQKFRG |
| | antibody | |
| | CDR-H2 | |
| 44 | SS1 | GGYDGRGFDY |
| | antibody | |
| | CDR-H3 | |
| 45 | SS1 antibody | 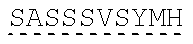 |
| | CDR-L1 | |
| 46 | SS1 antibody | 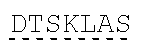 |
| | CDR-L2 | |
| 47 | SS1 antibody | 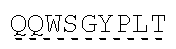 |
| | CDR-L3 | |
| 2 | anti- | MALPVTALLLPLALLLHAARPMECNWILPFILSVTSGVYSEILLQ |
| | mesothelin | QTGTVLARPGTSVKMSCKASGYTFTNYRMHWVKQRPGQGLE |
| | MGHmeso1 | WIGGIYPGNSDTNYNQKFKDKAKLTAVTSTSTANMELSSLTNE |
| | H/L 4-1BB | DSAVYYCLRGIRGSYFDYWGQGTTLTVSSGGGGSGGGGSGGG |
| | CAR | GSGGGGSMETDTILLWVLLLWVPGSTGDIVLTQSPASLAVSLGQ |
| | | RATISCKASQSVDYDGDSYMNWYQQKPGQPPKLLIYAASNLES |
| | | GIPARFSGSGSGTDFTLNIHPVEEEDAATYYCQQSNEDPSTFGGG |
| | | TKLEVKTTTPAPRPPTPAPTIASQPLSLRPEACRPAAGGAVHTRG |
| | | LDFACDIYIWAPLAGTCGVLLLSLVITLYCKRGRKKLLYIFKQPF |
| | | MRPVQTTQEEDGCSCRFPEEEEGGCELRVKFSRSADAPAYQQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPRSGGGGEGRGSLLTCGDVEENPGPRMVSKGEE |
| | | DNMAIIKEFMRFKVHMEGSVNGHEFEIEGEGEGRPYEGTQTAK |
| | | LKVTKGGPLPFAWDILSPQFMYGSKAYVKHPADIPDYLKLSFPE |
| | | GFKWERVMNFEDGGVVTVTQDSSLQDGEFIYKVKLRGTNFPSD |
| | | GPVMQKKTMGWEASSERMYPEDGALKGEIKQRLKLKDGGHY |
| | | DAEVKTTYKAKKPVQLPGAYNVNIKLDITSHNEDYTIVEQYER |
| | | AEGRHSTGGMDELYK |
| 3 | anti- | MALPVTALLLPLALLLHAARPMETDTILLWVLLLWVPGSTGDIV |
| | mesothelin | LTQSPASLAVSLGQRATISCKASQSVDYDGDSYMNWYQQKPGQ |
| | MGHmeso1 | PPKLLIYAASNLESGIPARFSGSGSGTDFTLNIHPVEEEDAATYYC |
| | L/H 4-1BB | QQSNEDPSTFGGGTKLEVKGGGGSGGGGSGGGGSGGGGSMEC |
| | CAR | NWILPFILSVTSGVYSEILLQQTGTVLARPGTSVKMSCKASGYTF |
| | | TNYRMHWVKQRPGQGLEWIGGIYPGNSDTNYNQKFKDKAKLT |
| | | AVTSTSTANMELSSLTNEDSAVYYCLRGIRGSYFDYWGQGTTL |
| | | TVSSTTTPAPRPPTPAPTIASQPLSLRPEACRPAAGGAVHTRGLD |
| | | FACDIYIWAPLAGTCGVLLLSLVITLYCKRGRKKLLYIFKQPFMR |
| | | PVQTTQEEDGCSCRFPEEEEGGCELRVKFSRSADAPAYQQGQN |
| | | QLYNELNLGRREEYDVLDKRRGRDPEMGGKPRRKNPQEGLYN |
| | | ELQKDKMAEAYSEIGMKGERRRGKGHDGLYQGLSTATKDTYD |
| | | ALHMQALPPRSGGGGEGRGSLLTCGDVEENPGPRMVSKGEEDN |
| | | MAIIKEFMRFK VHMEGSVNGHEFEIEGEGEGRPYEGTQTAKLK |
| | | VTKGGPLPFAWDILSPQFMYGSKAYVKHPADIPDYLKLSFPEGF |
| | | KWERVMNFEDGGVVTVTQDSSLQDGEFIYKVKLRGTNFPSDGP |
| | | VMQKKTMGWEASSERMYPEDGALKGEIKQRLKLKDGGHYDA |
| | | EVKTTYKAKKPVQLPGAYNVNIKLDITSHNEDYTIVEQYERAEG |
| | | RHSTGGMDELYK |
| 4 | anti- | MALPVTALLLPLALLLHAARPMECNWILPFILSVTSGVYSEILLQ |
| | mesothelin | QTGTVLARPGTSVKMSCKASGYTFTNYRMHWVKQRPGQGLE |
| | MGHmeso1 | WIGGIYPGNSDTNYNQKFKDKAKLTAVTSTSTANMELSSLTNE |
| | H/L CD28 | DSAVYYCLRGIRGSYFDYWGQGTTLTVSSGGGGSGGGGSGGG |
| | CAR | GSGGGGSMETDTILLWVLLLWVPGSTGDIVLTQSPASLAVSLGQ |
| | | RATISCKASQSVDYDGDSYMNWYQQKPGQPPKLLIYAASNLES |
| | | GIPARFSGSGSGTDFTLNIHPVEEEDAATYYCQQSNEDPSTFGGG |
| | | TKLEVKTTTPAPRPPTPAPTIASQPLSLRPEACRPAAGGAVHTRG |
| | | LDFACDIYIWAPLAGTCGVLLLSL VITLYCRSKRSRLLHSDYMN |
| | | MTPRRPGPTRKHYQPYAPPRDFAAYRSRVKFSRSADAPAYQQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPRSGGGGEGRGSLLTCGDVEENPGPRMVSKGEE |
| | | DNMAIIKEFMRFK VHMEGSVNGHEFEIEGEGEGRPYEGTQTAK |
| | | LKVTKGGPLPFAWDILSPQFMYGSKAYVKHPADIPDYLKLSFPE |
| | | GFKWERVMNFEDGGVVTVTQDSSLQDGEFIYKVKLRGTNFPSD |
| | | GPVMQKKTMGWEASSERMYPEDGALKGEIKQRLKLKDGGHY |
| | | DAEVKTTYKAKKPVQLPGAYNVNIKLDITSHNEDYTIVEQYER |
| | | AEGRHSTGGMDELYK |
| 5 | anti- | MALPVTALLLPLALLLHAARPMETDTILLWVLLLWVPGSTGDIV |
| | mesothelin | LTQSPASLAVSLGQRATISCKASQSVDYDGDSYMNWYQQKPGQ |
| | MGHmeso1 | PPKLLIYAASNLESGIPARFSGSGSGTDFTLNIHPVEEEDAATYYC |
| | L/H CD28 | QQSNEDPSTFGGGTKLEVKGGGGSGGGGSGGGGSGGGGSMEC |
| | CAR | NWILPFILSVTSGVYSEILLQQTGTVLARPGTSVKMSCKASGYTF |
| | | TNYRMHWVKQRPGQGLEWIGGIYPGNSDTNYNQKFKDKAKLT |
| | | AVTSTSTANMELSSLTNEDSAVYYCLRGIRGSYFDYWGQGTTL |
| | | TVSSTTTPAPRPPTPAPTIASQPLSLRPEACRPAAGGAVHTRGLD |
| | | FACDIYIWAPLAGTCGVLLLSLVITLYCRSKRSRLLHSDYMNMT |
| | | PRRPGPTRKHYQPYAPPRDFAAYRSRVKFSRSADAPAYQQGQN |
| | | QLYNELNLGRREEYDVLDKRRGRDPEMGGKPRRKNPQEGLYN |
| | | ELQKDKMAEAYSEIGMKGERRRGKGHDGLYQGLSTATKDTYD |
| | | ALHMQALPPRSGGGGEGRGSLLTCGDVEENPGPRMVSKGEEDN |
| | | MAIIKEFMRFKVHMEGSVNGHEFEIEGEGEGRPYEGTQTAKLK |
| | | VTKGGPLPFAWDILSPQFMYGSKAYVKHPADIPDYLKLSFPEGF |
| | | KWERVMNFEDGGVVTVTQDSSLQDGEFIYKVKLRGTNFPSDGP |
| | | VMQKKTMGWEASSERMYPEDGALKGEIKQRLKLKDGGHYDA |
| | | EVKTTYKAKKPVQLPGAYNVNIKLDITSHNEDYTIVEQYERAEG |
| | | RHSTGGMDELYK |
| 6 | Sibrotuzum | QVQLVQSGAEVKKPGASVKVSCKTSRYTFTEYTIHWVRQAPGQ |
| | ab VH | RLEWIGGINPNNGIPNYNQKFKGRVTITVDTSASTAYMELSSLRS |
| | | EDTAVYYCARRRIAYGYDEGHAMDYWGQGTLVTVSS |
| 7 | Sibrotuzum | DIVMTQSPDSLAVSLGERATINCKSSQSLLYSRNQKNYLAWYQ |
| | ab VL | QKPGQPPKLLIFWASTRESGVPDRFSGSGFGTDFTLTISSLQAED |
| | | VAVYYCQQYFSYPLTFGQGTKVEIK |
| 8 | Sibrotuzum | DIVMTQSPDSLAVSLGERATINCKSSQSLLYSRNQKNYLAWYQ |
| | ab sequence | QKPGQPPKLLIFWASTRESGVPDRFSGSGFGTDFTLTISSLQAED |
| | including | VAVYYCQQYFSYPLTFGQGTKVEIKGGGGSGGGGSGGGGSQV |
| | linker | QLVQSGAEVKKPGASVKVSCKTSRYTFTEYTIHWVRQAPGQRL |
| | | EWIGGINPNNGIPNYNQKFKGRVTITVDTSASTAYMELSSLRSED |
| | | TAVYYCARRRIAYGYDEGHAMDYWGQGTLVTVSS |
| 9 | CD3 | DIKLQQSGAELARPGASVKMSCKTSGYTFTRYTMHWVKQRPG |
| | antibody | QGLEWIGYINPSRGYTNYNQKFKDKATLTTDKSSSTAYMQLSSL |
| | CDR-VH | TSEDSAVYYCARYYDDHYCLDYWGQGTTLTVSSVE |
| 10 | CD3 | VDDIQLTQSPAIMSASPGEKVTMTCRASSSVSYMNWYQQKSGT |
| | antibody | SPKRWIYDTSKVASGVPYRFSGSGSGTSYSLTISSMEAEDAATY |
| | CDR-VL | YCQQWSSNPLTFGAGTKLELK |
| 11 | GARP | DIQMTQSPSSLSASLGDRVTITCQASQSISSYLAWYQQKPGQAP |
| | camelid | NILIYGASRLKTGVPSRFSGSGSGTSFTL |
| | antibody | TISGLEAEDAGTYYCQQYASVPVTFGQGTKVELK |
| 12 | LAP | MKLWLNWIFLVTLLNDIQCEVKL VESGGGLVQPGGSLSLSCAA |
| | antibody | SGFTFTDYYMSWVRQPPGKALEWLGFIRNKPNGYTTEYSASVK |
| | scFv (H-L) | GRFTISRDNSQSILYLQMNVLRAEDSATYYCARYTGGGYFDYW |
| | | GQGTTLTVSSGGGGSGGGGSGGGGSGGGGSMMSSAQFLGLLLL |
| | | CFQGTRCDIQMTQTTSSLSASLGDRLTISCRASQDISNYLNWYQ |
| | | QKPDGTVKLLIYYTSRLHSGVPSRFSGSGSGTDYSLTISNLEQAD |
| | | IATYFCQQGDTLPWTFGGGTKLEIK |
| 13 | MGHmeso1 | NYRMH |
| | antibody | |
| | CDR-H1 | |
| 14 | MGHmeso1 | GIYPGNSDTNYNQKFKD |
| | antibody | |
| | CDR-H2 | |
| 15 | MGHmeso1 | GIRGSYFDY |
| | antibody | |
| | CDR-H3 | |
| 16 | MGHmeso1 | KASQSVDYDGDSYMN |
| | antibody | |
| | CDR-L1 | |
| 17 | MGHmeso1 | AASNLES |
| | antibody | |
| | CDR-L2 | |
| 18 | MGHmeso1 | QQSNEDPST |
| | antibody | |
| | CDR-L3 | |
| 19 | MGHmeso1 | MECNWILPFILSVTSGVYSEILLQQTGTVLARPGTSVKMSCKAS |
| | antibody | GYTFTNYRMHWVKQRPGQGLEWIGGIYPGNSDTNYNQKFKDK |
| | VH | AKLTAVTSTSTANMELSSLTNEDSAVYYCLRGIRGSYFDYWGQ |
| | | GTTLTVSS |
| 20 | MGHmeso1 | METDTILLWVLLLWVPGSTGDIVLTQSPASLAVSLGQRATISCK |
| | antibody VL | ASQSVDYDGDSYMNWYQQKPGQPPKLLIYAASNLESGIPARFS |
| | | GSGSGTDFTLNIHPVEEEDAATYYCQQSNEDPSTFGGGTKLEVK |
| 21 | MGHmeso1 | MECNWILPFILSVTSGVYSEILLQQTGTVLARPGTSVKMSCKAS |
| | anti- | GYTFTNYRMHWVKQRPGQGLEWIGGIYPGNSDTNYNQKFKDK |
| | mesothelin | AKLTAVTSTSTANMELSSLTNEDSAVYYCLRGIRGSYFDYWGQ |
| | antibody | GTTLTVSSGGGGSGGGGSGGGGSGGGGSMETDTILLWVLLLW |
| | VH-VL | VPGSTGDIVLTQSPASLAVSLGQRATISCKASQSVDYDGDSYMN |
| | | WYQQKPGQPPKLLIYAASNLESGIPARFSGSGSGTDFTLNIHPVE |
| | | EEDAATYYCQQSNEDPSTFGGGTKLEVK |
| 22 | MGHmeso1 | METDTILLWVLLLWVPGSTGDIVLTQSPASLAVSLGQRATISCK |
| | anti- | ASQSVDYDGDSYMNWYQQKPGQPPKLLIYAASNLESGIPARFS |
| | mesothelin | GSGSGTDFTLNIHPVEEEDAATYYCQQSNEDPSTFGGGTKLEVK |
| | antibody | GGGGSGGGGSGGGGSGGGGSMECNWILPFILSVTSGVYSEILLQ |
| | VL-VH | QTGTVLARPGTSVKMSCKASGYTFTNYRMHWVKQRPGQGLE |
| | | WIGGIYPGNSDTNYNQKFKDKAKLTAVTSTSTANMELSSLTNE |
| | | DSAVYYCLRGIRGSYFDYWGQGTTLTVSS |
| 23 | mesothelin | MALPTARPLLGSCGTPALGSLLFLLFSLGWVQPSRTLAGETGQE |
| | protein | AAPLDGVLANPPNISSLSPRQLLGFPCAEVSGLSTERVRELAVAL |
| | sequence | AQKNVKLSTEQLRCLAHRLSEPPEDLDALPLDLLLFLNPDAFSG |
| | | PQACTRFFSRITKANVDLLPRGAPERQRLLPAALACWGVRGSLL |
| | | SEADVRALGGLACDLPGRFVAESAEVLLPRLVSCPGPLDQDQQ |
| | | EAARAALQGGGPPYGPPSTWSVSTMDALRGLLPVLGQPIIRSIPQ |
| | | GIVAAWRQRSSRDPSWRQPERTILRPRFRREVEKTACPSGKKAR |
| | | EIDESLIFYKKWELEACVDAALLATQMDRVNAIPFTYEQLDVLK |
| | | HKLDELYPQGYPESVIQHLGYLFLKMSPEDIRKWNVTSLETLKA |
| | | LLEVNKGHEMSPQAPRRPLPQVATLIDRFVKGRGQLDKDTLDT |
| | | LTAFYPGYLCSLSPEELSSVPPSSIWAVRPQDLDTCDPRQLDVLY |
| | | PKARLAFQNMNGSEYFVKIQSFLGGAPTEDLKALSQQNVSMDL |
| | | ATFMKLRTDAVLPLTVAEVQKLLGPHVEGLKAEERHRPVRDWI |
| | | LRQRQDDLDTLGLGLQGGIPNGYLVLDLSMQEALSGTPCLLGP |
| | | GPVLTVLALLLASTLA |
| 24 | CD8 | TTTPAPRPPTPAPTIASQPLSLRPEACRPAAGGAVHTRGLDFACD |
| | hinge/TM | IYIWAPLAGTCGVLLLSLVITLYC |
| | domain | |
| 38 | CD28 | TTTPAPRPPTPAPTIASQPLSLRPEACRPAAGGAVHTRGLDFACD |
| | hinge/TM | IYIWAPLAGTCGVLLLSL VITLYC |
| | domain | |
| 39 | Nucleic acid | ACCACTACCCCAGCACCGAGGCCACCCACCCCGGCTCCTACC |
| | sequence | ATCGCCTCCCAGCCTCTGTCCCTGCGTCCGGAGGCATGTAGA |
| | encoding | CCCGCAGCTGGTGGGGCCGTGCATACCCGGGGTCTTGACTTC |
| | CD28 | GCCTGCGATATCTACATTTGGGCCCCTCTGGCTGGTACTTGCG |
| | hinge/TM | GGGTCCTGCTGCTTTCACTCGTGATCACTCTTTACTGT |
| | domain | |
| 25 | 4-1 BB ICD | KRGRKKLLYIFKQPFMRPVQTTQEEDGCSCRFPEEEEGGCEL |
| 26 | CD3-zeta | RVKFSRSADAPAYQQGQNQLYNELNLGRREEYDVLDKRRGRD |
| | ICD-1 | PEMGGKPRRKNPQEGLYNELQKDKMAEAYSEIGMKGERRRGK |
| | | GHDGLYQGLSTATKDTYDALHMQALPPR |
| 27 | CD3-zeta | RVKFSRSADAPAYQQGQNQLYNELNLGRREEYDVLDKRRGRD |
| | ICD-2 | PEMGGKPRRKNPQEGLYNELQKDKMAEAYSEIGMKGERRRGK |
| | | GHDGLYQGLSTATKDTYDALHMQALPPR |
| 28 | CD28 ICD | RSKRSRLLHSDYMNMTPRRPGPTRKHYQPYAPPRDFAAYRS |
| 29 | CD8 leader | MALPVTALLLPLALLLHAARP |
| 30 | IgK signal | METDTLLLWVLLLWVPGSTGD |
| | sequence | |
| 31 | linker-1 | GGGSGGGSGGGS |
| | (G3S)3 | |
| 32 | linker-2 | GGGGSGGGGSGGGGS |
| | (G4S)3 | |
| 33 | linker-3 | GGGGSGGGGSGGGGSGGGGS |
| | (G4S)4 | |
| 34 | linker-4 | GSTSGSGKPGSGEGSTKG |
| 35 | linker-5 | GGSSRSSSSGGGGSGGGG |
| 36 | T2A | SGGGGEGRGSLLTCGDVEENPGPR |
| 37 | anti-CD19 | DIQLTQSPASLAVSLGQRATISCKASQSVDYDGDSYLNWYQQIP |
| | scFv | GQPPKLLIYDASNLVSGIPPRFSGSGSGTDFTLNIHPVEKVDAAT |
| | | YHCQQSTEDPWTFGGGTKLEIKGGGGSGGGGSGGGGSQVQLQ |
| | | QSGAELVRPGSSVKISCKASGYAFSSYWMNWVKQRPGQGLEWI |
| | | GQIWPGDGDTNYNGKFKGKATLTADESSSTAYMQLSSLASEDS |
| | | AVYFCARRETTTVGRYYYAMDYWGQGTTVTVSS |

### Administration and Treatment Subject

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., a pancreatic cancer, a lung cancer, an ovarian cancer, endometrial cancer, biliary cancer, gastric cancer, or mesothelioma or another type of cancer expressing mesothelin, among others) or one or more complications related to such a condition, and optionally, have already undergone treatment for the condition or the one or more complications related to the condition.

Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

### Pharmaceutical Compositions

As used herein, the term “pharmaceutical composition” refers to the active agent in combination with a pharmaceutically acceptable carrier e.g. a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

In one aspect of the technology, the technology described herein relates to a pharmaceutical composition including activated CART cells as described herein, and optionally a pharmaceutically acceptable carrier. The active ingredients of the pharmaceutical composition at a minimum include activated CART cells as described herein. In some embodiments, the active ingredients of the pharmaceutical composition consist essentially of activated CART cells as described herein. In some embodiments, the active ingredients of the pharmaceutical composition consist of activated CAR T cells as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier”, “pharmaceutically acceptable excipient” or the like are used interchangeably herein.

In some embodiments, the pharmaceutical composition including activated CAR T cells as described herein can be a parenteral dose form. Since administration of parenteral dosage forms typically bypasses the patient's natural defenses against contaminants, the components apart from the CART cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CART cells preparation prior to administration. Suitable vehicles that can be used to provide parenteral dosage forms of activated CAR T cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

“Unit dosage form” as the term is used herein refers to a dosage for suitable one administration. By way of example, a unit dosage form can be an amount of therapeutic disposed in a delivery device, e.g., a syringe or intravenous drip bag. In some embodiments, a unit dosage form is administered in a single administration. In another, embodiment more than one unit dosage form can be administered simultaneously.

In some embodiments, the activated CAR T cells described herein are administered as a monotherapy, i.e., another treatment for the condition is not concurrently administered to the subject. A pharmaceutical composition including the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. Med. 30 319:1676, 1988).

In certain aspects, it may be desired to administer activated CART cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 35 10 cc to 400 cc. In certain aspects, T cells are activated from blood draws of 20 cc, 30 cc, 40 cc, 50 cc, 60cc, 70cc, 80cc, 90cc, or 100cc.

### Administration

In some embodiments, the methods described herein relate to treating a subject having or diagnosed as having cancer, a plasma cell disease or disorder, or an autoimmune disease or disorder with a mammalian cell including any of the CAR polypeptides (and optional antibody reagents or cytokines) described herein, or a nucleic acid encoding any of the CAR polypeptides (and optional antibody reagents or cytokines) described herein. The CART cells described herein include mammalian cells including any of the CAR polypeptides (and optional antibody reagents or cytokines) described herein, or a nucleic acid encoding any of the CAR polypeptides (and optional antibody reagents or cytokines) described herein. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein include administering an effective amount of activated CAR T cells described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In some embodiments, the compositions described herein are administered systemically or locally. In a preferred embodiment, the compositions described herein are administered intravenously. In another embodiment, the compositions described herein are administered at the site of a tumor.

The term “effective amount” as used herein refers to the amount of activated CAR T cells needed to alleviate at least one or more symptom of the disease or disorder, and relates to a sufficient amount of the cell preparation or composition to provide the desired effect. The term “therapeutically effective amount” therefore refers to an amount of activated CART cells that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CART cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

### Modes of Administration

Modes of administration can include, for example intravenous (iv) injection or infusion. The compositions described herein can be administered to a patient transarterially, intratumorally, intranodally, intraperitoneally or intramedullary. In some embodiments, the compositions of T cells may be injected directly into a tumor, lymph node, or site of infection. In some embodiments, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an artificial APC, e.g., an aAPC expressing anti-CD28 and anti-CD3 CD Rs, and treated such that one or more CAR constructs of the technology may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells. In some embodiment, expanded cells are administered before or following surgery. In some embodiments, lymphodepletion is performed on a subject prior to administering one or more CART cell as described herein. In such embodiments, the lymphodepletion can include administering one or more of melphalan, cytoxan, cyclophosphamide, and fludarabine. The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combination Therapy

The activated CART cells described herein can optionally be used in combination with each other and with other known agents and therapies, as can determined to be appropriate by those of skill in the art. In one example, two or more CART cells targeting different Treg markers (e.g., GARP, LAP, CD25, and cytotoxic T lymphocyte-associated antigen-4 (CTLA-4) etc.) can be administered in combination. In another example, two or more CART cells targeting different cancer antigens are administered in combination. In a further example, one or more CART cell targeting a Treg marker (e.g., GARP, LAP, etc.) and one or more CART cell targeting one or more tumor antigens are administered in combination.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The activated CART cells described herein and the at least one additional therapeutic agent can be administered simultaneously, in the same or in separate compositions, or sequentially. For sequential administration, the CAR expressing cell described herein can be administered first, and the additional agent can be administered second, or the order of administration can be reversed. The CAR T therapy and/or other therapeutic agents, procedures or modalities can be administered during periods of active disorder, or during a period of remission or less active disease. The CART therapy can be administered before another treatment, concurrently with the treatment, post-treatment, or during remission of the disorder.

When administered in combination, the activated CART cells and the additional agent (e.g., second or third agent), or all, can be administered in an amount or dose that is higher, lower or the same as the amount or dosage of each agent used individually, e.g., as a monotherapy. In certain embodiments, the administered amount or dosage of the activated CART cells, the additional agent (e.g., second or third agent), or all, is lower (e.g., at least 20%, at least 30%, at least 40%, or at least 50%) than the amount or dosage of each agent used individually. In other embodiments, the amount or dosage of the activated CART cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the activated CART cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al., J. Neurosurg. 108:963-971, 2008.

In some embodiments, the activated CART cells described herein can be used in combination with a checkpoint inhibitor. Exemplary checkpoint inhibitors include anti-PD-1 inhibitors (Nivolumab, MK-3475, Pembrolizumab, Pidilizumab, AMP-224, AMP-514), anti-CTLA4 inhibitors (Ipilimumab and Tremelimumab), anti-POL 1 inhibitors (Atezolizumab, Avelomab, MSB0010718C, MED14736, and MPDL3280A), and anti-TIM3 inhibitors.

In some embodiments, the activated CART cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNUR); Cisplatin (also known as CDDP, Platinol® and Platinol®-AO); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (IR,2R,45)-4-[(2R)-2 [(1R,95, 125, 15R, 16E, 18R, 19R,21 R,235,24E,26E,28Z,305,325,35R)-I, 18-dihydroxy-19,30-dimethoxy-15, 17,21,23, 29,35-hexamethyl-2,3, 10, 14,20-pentaoxo-1 1,36-dioxa-4-azatricyclo[30.3.1.04′9] hexatriaconta-16,24,26,28-tetraen-12-yl]propyl]-2-methoxycyclohexyl dimethylphosphinate, also known AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis [(35)-3-methylmorpholin-4-yl]pyrido[2,3-(i]pyrimidin-7-yl}-2-methoxyphenyl) methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7 (8H)-one (PF04691502, CAS 1013101-36-4); and N2-[1,4-dioxo-4-[[4-(4-oxo-8-phenyl-4H-lbenzopyran-2-yl) morpholin ium-4-yl]methoxy]butyl]-L-arginylglycyl-L-α-aspartyl L-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, ldamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2- yl)amino)-1-oxo-3-phenylpropan-2-yl)-2-((5)-2-(2-morphol inoacetamido)-4-phenylbutanamido)-pentanamide); marizom ib(NPT0052); ixazomib citrate (MLN-9708); delanzomib (CEP-18770); and O-Methyl-N-[(2-methyl-5-thiazolyl) carbonyl]-L-seryl-O-methyl-N-[(I IS′)-2-[(2 R)-2-methyl-2-oxi ranyl]-2-oxo-1-(phenyl methyl)ethyl]-L-serinamide (ONX-0912).

One of skill in the art can readily identify a chemotherapeutic agent of use (e.g., see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chapters 28-29 in Abeloff's Clinical Oncology, 2013 Elsevier; and Fischer D.S. (ed.): The Cancer Chemotherapy Handbook, 4th ed. St. Louis, Mosby-Year Book, 2003).

In an embodiment, activated CAR T cells described herein are administered to a subject in combination with a molecule that decreases the level and/or activity of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

### Efficacy

The efficacy of activated CART cells in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate.

Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### Cancers

In some embodiments, the immune cells (e.g., T cells) including a CAR, such as a CART TEAM described herein, can also be used to treat a cancer having heterogeneous antigen expression (e.g. mesothelin expression). For example, the CAR component of the CART TEAM construct can include an extracellular target binding domain that binds to one antigen expressed by the cancer, while the TEAM component of the CART TEAM construct can bind a second antigen expressed by the cancer in addition to a T cell antigen (e.g., CD3). “Cancer” as used herein can refer to a hyperproliferation of cells whose unique trait, loss of normal cellular control, results in unregulated growth, lack of differentiation, local tissue invasion, and metastasis. Exemplary cancers include, but are not limited to, glioblastoma, prostate cancer, glioma, leukemia, lymphoma, multiple myeloma, or a solid tumor, e.g., lung cancer and pancreatic cancer. Nonlimiting examples of leukemia include acute myeloid leukemia (AML), chronic myeloid leukemia (CML), acute lymphocytic leukemia (ALL), and chronic lymphocytic leukemia (CLL). In some embodiments, the cancer is ALL or CLL. Non-limiting examples of lymphoma include diffuse large B-cell lymphoma (DLBCL), follicular lymphoma, small lymphocytic lymphoma (SLL), mantle cell lymphoma (MCL), marginal zone lymphomas, Burkitt's lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)). In some embodiments, the cancer is DLBCL or follicular lymphoma. Non-limiting examples of solid tumors include adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor.

Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

Cluster of differentiation (CD) molecules are cell surface markers present on leukocytes. As a leukocyte differentiates and matures its CD profile changes. In the case that a leukocytes turns into a cancer cell (i.e., a lymphoma), its CD profile is important in diagnosing the disease. The treatment and prognosis of certain types of cancers is reliant on determining the CD profile of the cancer cell. “COX+”, wherein “X” is a CD marker, indicates the CD marker is present in the cancer cell, while “COX-” indicates the marker is not present. One skilled in the art will be capable of assessing the CD molecules present on a cancer cell using standard techniques, for example, using immunofluorescence to detect commercially available antibodies bound to the CD molecules.

### Autoimmune Disorders

As used herein, an “autoimmune disease or disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, acute disseminated encephalomyelitis, antiphospholipid antibody syndrome, aplastic anemia, idiopathic thrombocytopenia purpura, myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM). In some embodiments, the mammalian cell is obtained for a patient having an immune system disorder that results in abnormally low activity of the immune system, or immune deficiency disorders, which hinders one's ability to fight a foreign agent (e.g., a virus or bacterial cell). A plasma cell is a white blood cell produces from B lymphocytes which function to generate and release antibodies needed to fight infections. As used herein, a “plasma cell disorder or disease” is characterized by abnormal multiplication of a plasma cell. Abnormal plasma cells are capable of “crowding out” healthy plasma cells, which results in a decreased capacity to fight a foreign object, such as a virus or bacterial cell. Non-limiting examples of plasma cell disorders include amyloidosis, Waldenstrom's macroglobulinemia, osteosclerotic myeloma (POEMS syndrome), monoclonal gammopathy of unknown significance (MGUS), and plasma cell myeloma.

### CAR T Cell Therapy

One aspect of the technology described herein relates to a method of treating cancer, a plasma cell disorder, or an autoimmune disease in a subject in need thereof, the method including: engineering a T cell to include any of the CAR polypeptides (and optional antibody reagents or cytokines) described herein on the T cell surface; and administering to a subject a CAR T cell as described herein. In the case of cancer, the method can be for treating diagnosed cancer, preventing recurrence of cancer, or for use in an adjuvant or neoadjuvant setting. In some embodiments, the subject has a cancer that expresses mesothelin.

One aspect of the technology described herein relates to a method of treating cancer, a plasma cell disorder, or an autoimmune disease in a subject in need thereof, the method including: administering the cell of any of the mammalian cells including the any of the CAR polypeptides (and optional antibody reagents or cytokines) described herein. In some embodiments of any of aspect, the engineered CAR-T cell is stimulated and/or activated prior to administration to the subject.

In some embodiments, the immune cells (e.g., T cells) including a CAR, such as a CART TEAM described herein, can be used to treat cancer expression mesothelin, e.g., a pancreatic cancer, a lung cancer, an ovarian cancer, endometrial cancer, biliary cancer, gastric cancer, or mesothelioma. In some embodiments, the cancer expresses mesothelin. In some embodiments, the CAR T cells or a pharmaceutical composition thereof are administered to a subject who has a cancer as described above. In some embodiments, the CAR T cells administered to a subject who has cancer comprise an anti-mesothelin CAR. In some embodiments, the CAR T cells administered to a subject who has cancer comprise an anti-mesothelin CAR and a FAP TEAM. In some embodiments, the CAR T cells administered to a subject who has cancer comprise a polypeptide comprising the amino acid sequence of any one of SEQ ID NOs: 1-5.

In some embodiments, the immune cells (e.g., T cells) comprising a CAR, such as a CAR T TEAM described herein, can be used to prevent or reduce immunosuppression due to, e.g., Tregs, in the tumor microenvironment. Furthermore, such CAR T TEAMs are useful for preventing or reducing T cell exhaustion in the tumor microenvironment. In some embodiments, the CAR T cells or a pharmaceutical composition thereof are administered to a subject who having an autoimmune disorder described above. In some embodiments, the CAR T cells administered to a subject who has cancer comprise the amino acid sequence of any one of SEQ ID NOs: 1-5. In some embodiments, the CAR T cells administered to a subject who has an autoimmune disorder comprise an anti-mesothelin CAR. In some embodiments, the CAR T cells administered to a subject who has an autoimmune disorder comprise an anti-mesothelin CAR and a FAP TEAM. In some embodiments, the CAR T cells administered to a subject who has an autoimmune disorder comprise the amino acid sequence of any one of SEQ ID NOs: 1-5.

### EXAMPLES

### Example 1: Anti-Mesothelin CAR-T Cells for Treatment of Cancer

Mesothelin is a promising therapeutic target on numerous cancers, including pancreatic cancer. To target mesothelin expressing cancers, three different types of mesothelin chimeric antigen receptor (CAR) T cells were developed: type 1 expressing an SS1-BBz CAR, type 2 expressing an MGHmesol L/H-BBz CAR, and type 3 expressing an MGHmesol H/L-BBz CAR. Each anti-mesothelin CAR developed comprises a CD8 leader, a VH and a VL domain from a scFv mesothelin antibody, a CD8 hinge/transmembrane, a 4-1 BBz intracellular domain (ICD), a CD3 ICD, and a T2A. SS1-BBz comprises the VH and VL from the SS1 scFv antibody. MGHmesol L/H comprises the VH and VL from the MGHmesol scFv antibody where the VL domain is located N-terminal to the VH domain. MGHmesol H/L comprises the VH and VL from the MGHmesol scFv antibody where the VH domain is located N-terminal to the VL domain.

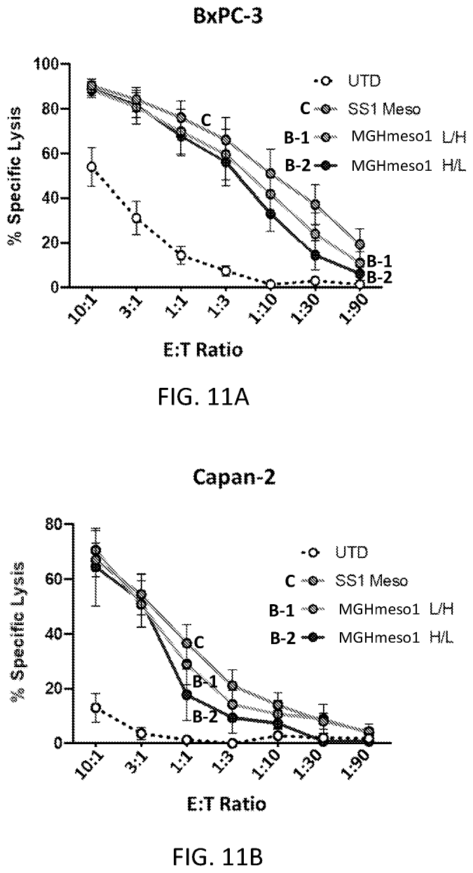

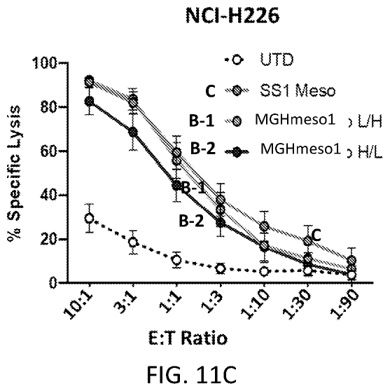

The cancer killing efficacy of developed anti-mesothelin CAR T cells was tested in three different cancer cells lines: BxPC-3 (pancreatic cancer), Capan-2 (pancreatic cancer), and NCI-H226 (lung cancer) (FIGS. 11A-11C). Results demonstrated that all three anti-mesothelin CAR T cells induced specific lysis of each type of cancer cell. MGHmesol L/H-41 BBz (comprising the L/H orientation anti-mesothelin CAR) consistently demonstrated specific lysis equal to or greater than MGHmesol H/L-41 BBz (comprising the H/L orientation anti-mesothelin CAR), so MGHmeso1L/H-41 BBz CAR T cells were selected for further study.

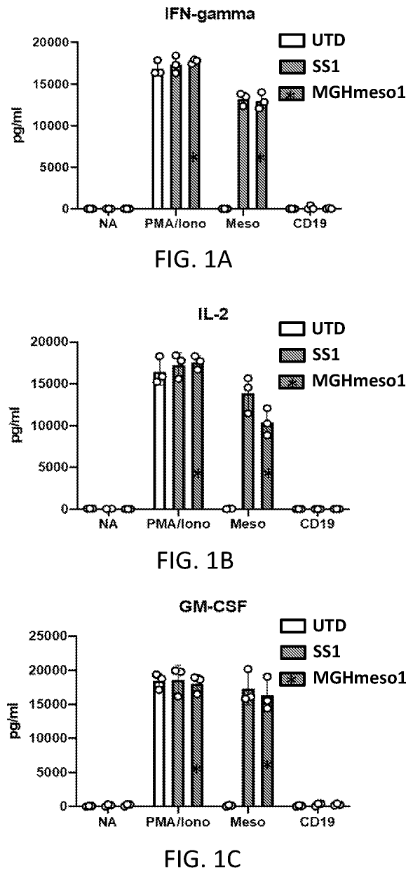

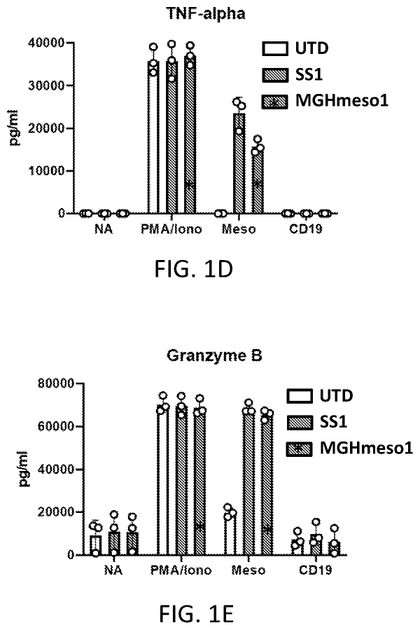

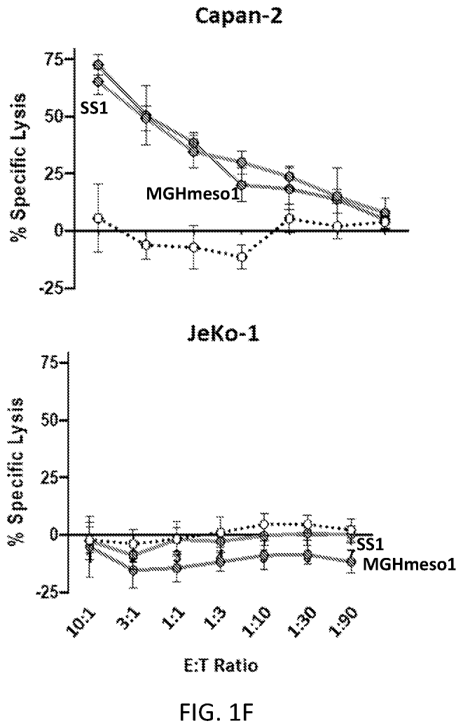

The cytokine expression profile of SS1-BBz and MGHmesol L/H-41 BBz CAR-T cells was further analyzed (FIGS. 1A-1E, n=3 replicates). Results showed that expression of the anti-mesothelin CARs (SS1-41 BBz and MGHmesol L/H-41 BBz) greatly increased expression of INF-gamma, IL-2, GM-CSF, TNF-alpha, and Granzyme B in the CAR T cells. Additionally, results showed that SS1-BBz and MGHmesol L/H-41BBz specifically lysed mesothelin positive Capan-2 cells, but did not lyse mesothelin negative JeKo-1 cells (Mantle cell lymphoma) (FIGS. 1E-1F). The results from FIG. 1 demonstrate that SS1-41BBz and MGHmesol L/H-41BBz cells exhibit similar cell function and cytotoxicity.

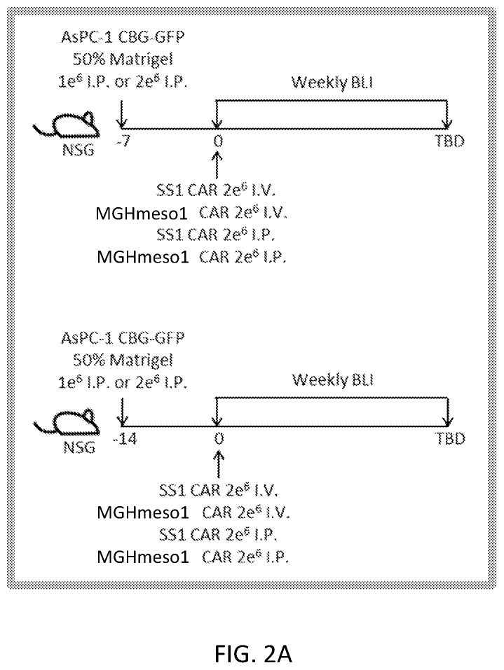

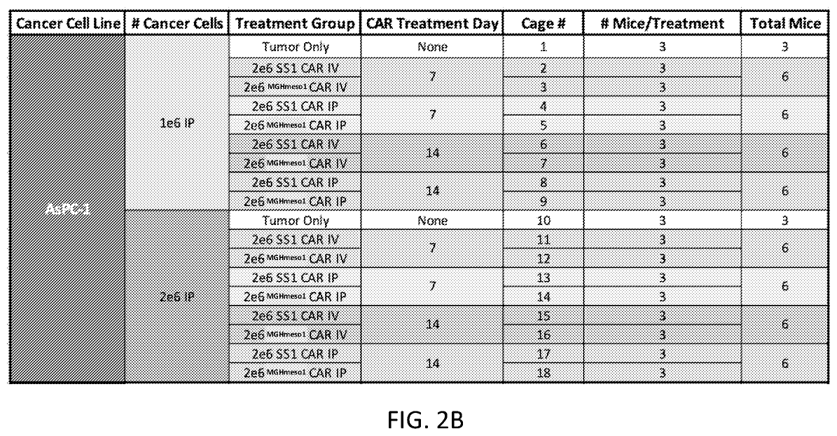

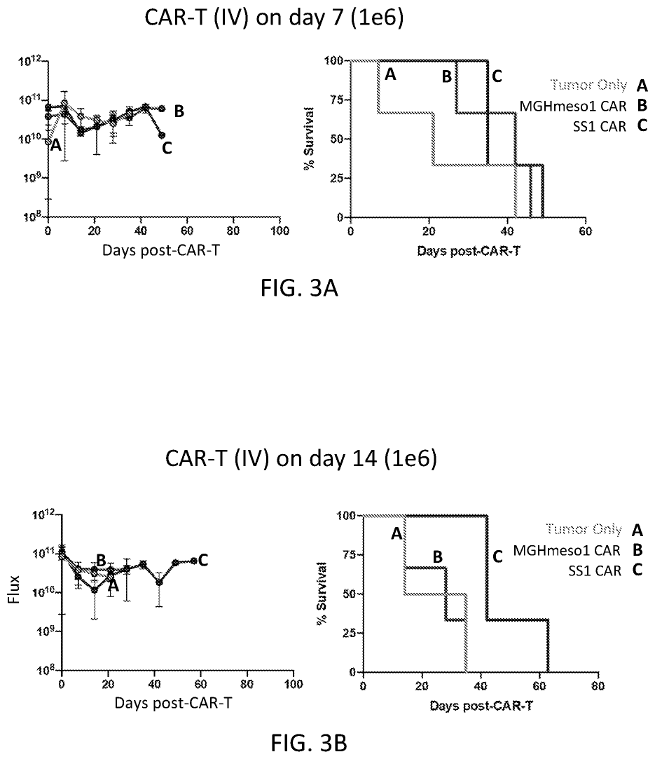

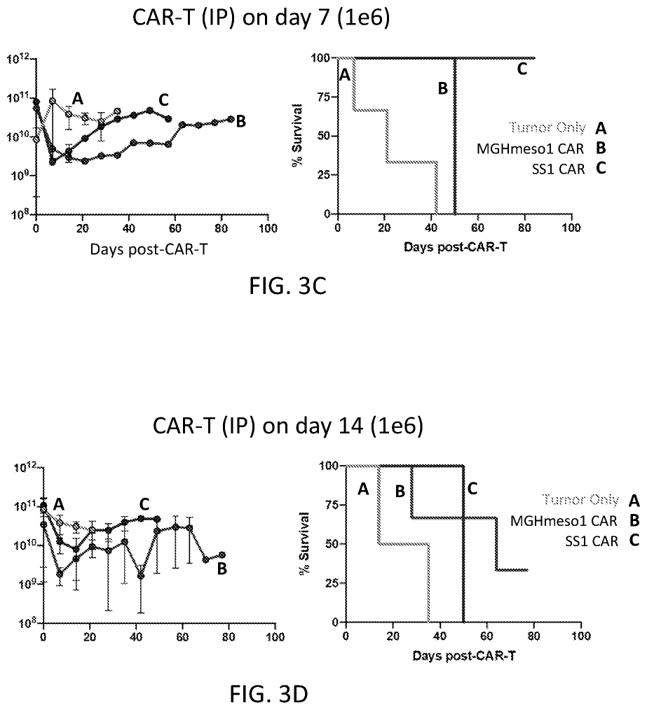

The efficacy of SS1-41BBz and MGHmesol L/H-41BBz CAR T cells was further tested in mice in vivo. FIGS. 2A-2B show the details of a series of experiments administering CAR T to mice, with the results of the series of experiments shown in FIGS. 3A-10D. CAR T cells were administered at different doses, at different times (7 or 14 days), and by different methods (intravenous (IV) and intraperitoneal injection (IP)) after mice were injected with AsPC-1 (pancreatic cancer) cells.

FIGS. 3A-3D compares administration of 1e6 SS1-BBz and MGHmesol L/H-BBz CAR-T cells to mice injected with AsPC-1. FIG. 3A compares the flux and percent survival after administration by IV at day 7 after treatment. Flux is defined as photons per second, and is a measure of tumor burden. The cancer cell lines tested have been transduced with luciferase and produce light upon injection with D-luciferin substrate. FIG. 3B compares the flux and percent survival after administration by IV at day 14 after treatment. FIG. 3C compares the flux and percent survival after administration by IP at day 7 after treatment. FIG. 3D compares the flux and percent survival after administration by IP at day 14 after treatment. Overall, results from FIGS. 3A-3D show that MGHmesol CAR T cells are more effective than the SS1 CAR T when administered by IP injection. These results demonstrate the potential utility and advantage of MGHmesol CAR T cells for treating a mesothelin-expressing cancer and that the mode of administration of said CAR T cells may be a factor in the relative efficacy of treatment.

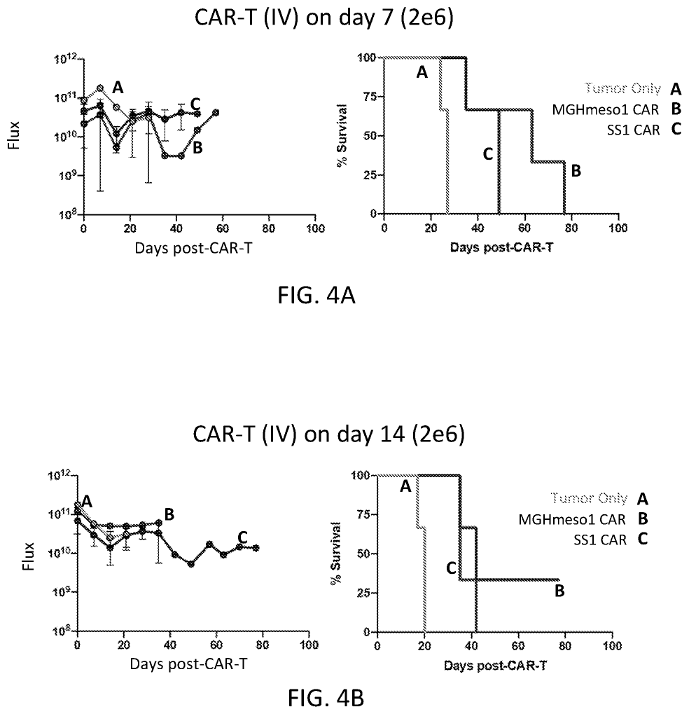

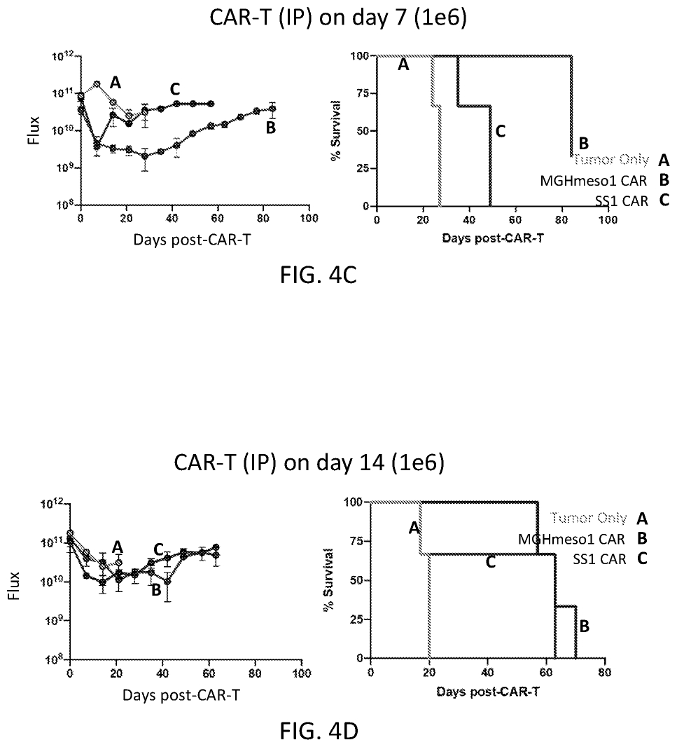

FIGS. 4A-4D also compare the flux and percent survival of mice injected with AsPC-1 and administered a higher dose (2e6 cells) of SS1-BBz or MGHmesol L/H-BBz CAR-T cells. Results from FIGS. 4A-4D show that IP administration of MGHmesol CAR T cells is more effective than IV administration and that MGHmesol CAR T cells have greater efficacy than SS1 CAR T cells when administered at day 7. These results further demonstrate the potential utility and advantage of MGHmesol CAR T cells for treating a mesothelin-expressing cancer and that both timing of administration as well as the mode of administration of said CAR T cells may be a factor in the relative efficacy of treatment.

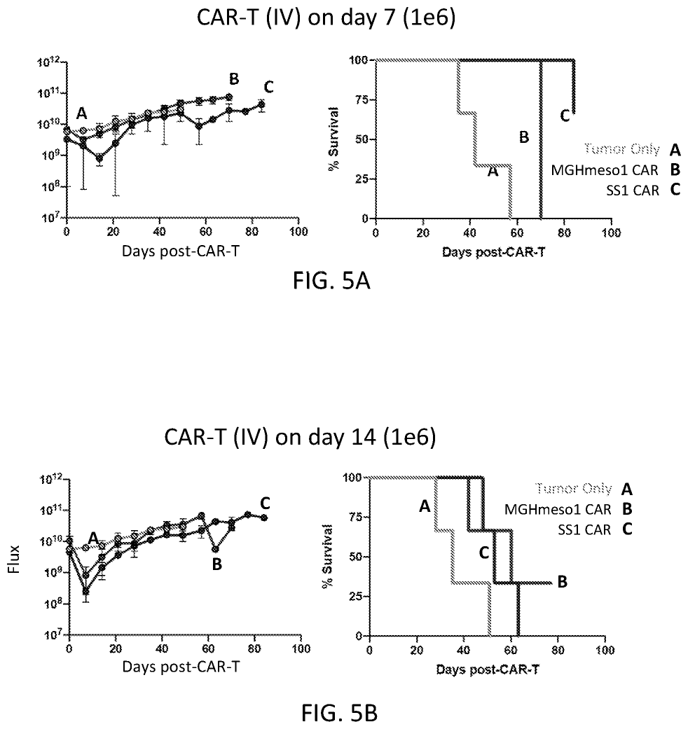

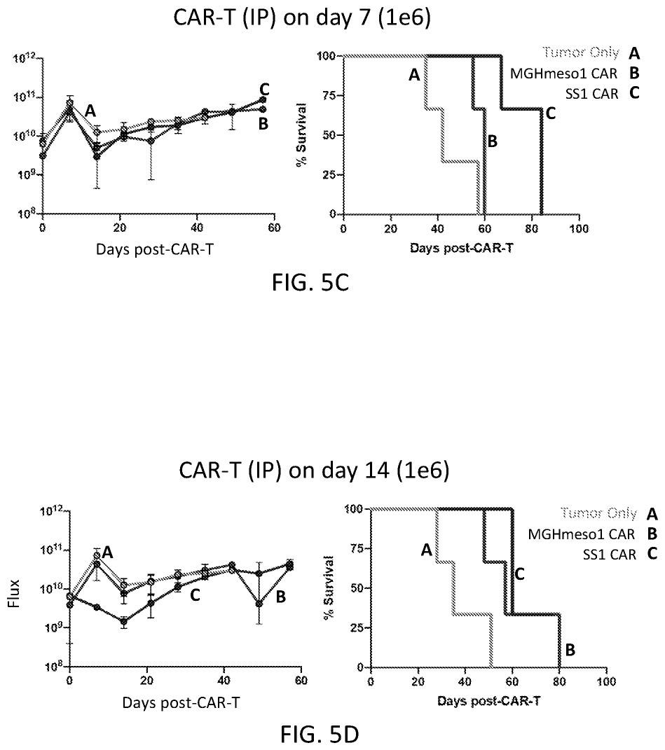

FIGS. 5A-5D compare the flux and percent survival of mice injected with a different line of pancreatic cancer cells (BXPC3) and administered SS1-BBz or MGHmesol L/H-BBz CAR T cells at a dose of 1e6 CAR T cells. Results from FIGS. 5A-5D show that IP administration of 1e6 MGHmesol CAR T cells is equally effective or more effective than IV administration when treating mice BXPC3 cancer in mice. Results from FIG. 5A-5D also show that SS1 CAR T cells and MGHmesol CAR T cells have similar efficacy at day 14 when treating BXPC3 cancer in mice, but SS1 CAR T cells have slightly increased efficacy at day 7.

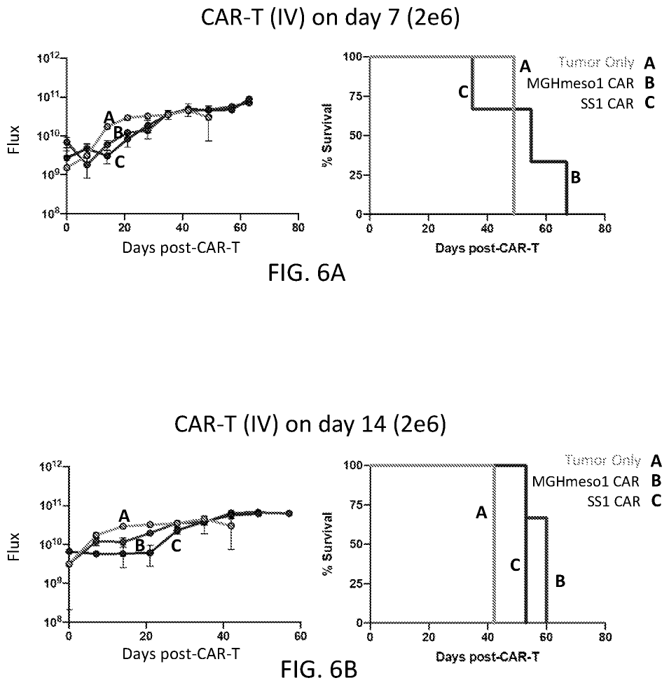

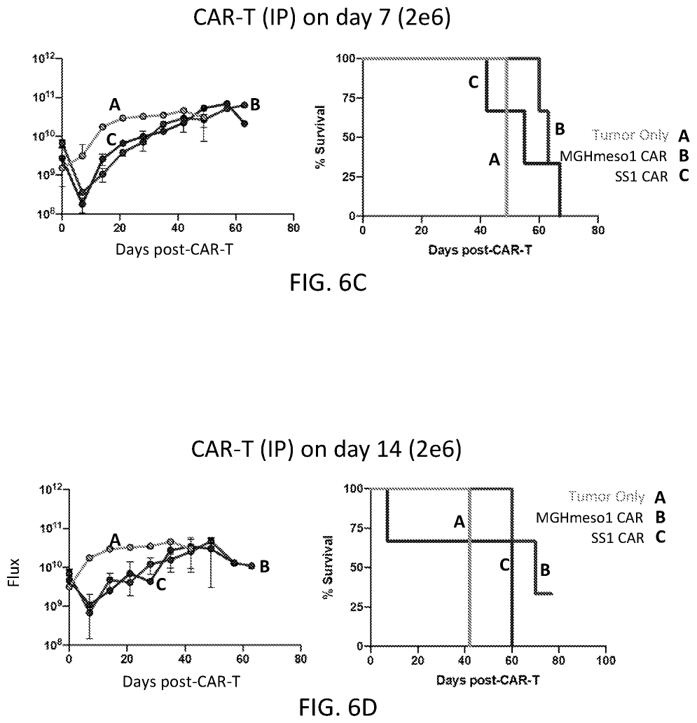

FIGS. 6A-6D compare the flux and percent survival of mice injected with BXPC3 pancreatic cancer cells and administered SS1-BBz or MGHmesol L/H-BBz CAR T cells at a dose of 2e6 CAR T cells. Results from FIGS. 6A-6D show that MGHmesol L/H CAR T cells have increased efficacy compared to SS1 Car T cells when administered by IP injection to mice with BXPC3 cancer at this higher dose relative to FIGS. 5A-5D. These results demonstrate the advantage of treating a mesothelin-expressing cancer with MGHmesol CAR T cells and suggest a dose dependent improvement over CAR T cells expressing a previously known CAR (SS1).

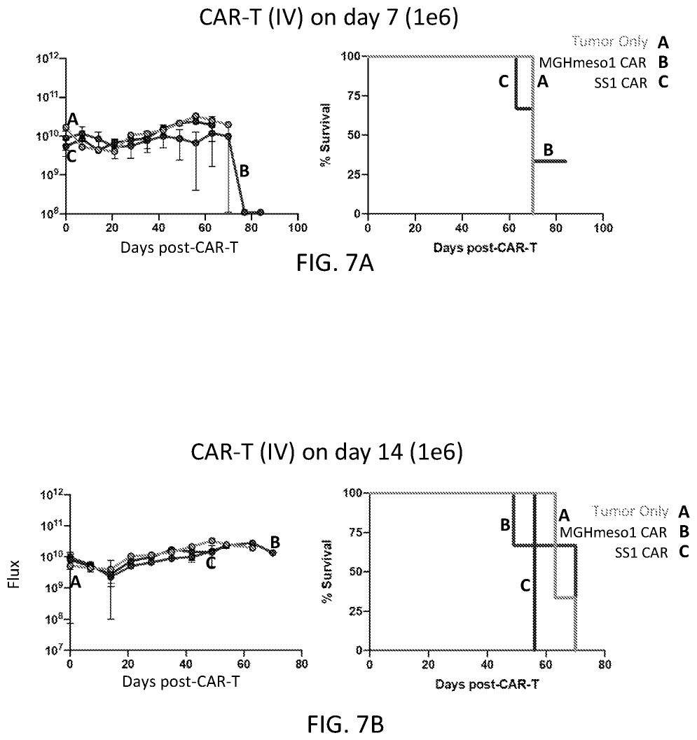

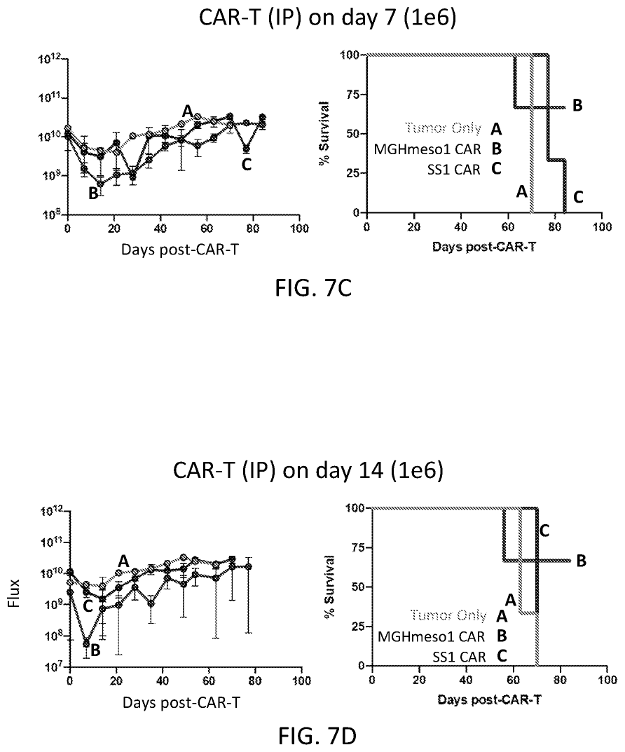

FIGS. 7A-7D compare the flux and percent survival of mice injected with a different line of pancreatic cancer cells (PANC1) and administered SS1-BBz or MGHmesol L/H-BBz CAR T cells at a dose of 1e6 CAR T cells. Results from FIGS. 7A-7D show that MGHmesol L/H CAR T cells have increased efficacy compared to SS1 Car T cells when administered by IP injection to mice with PANC1 cancer. These results demonstrate the potential utility and advantage of MGHmesol CAR T cells for treating another mesothelin-expressing cancer and that the mode of administration of said CAR T cells may be a factor in the relative efficacy of treatment.

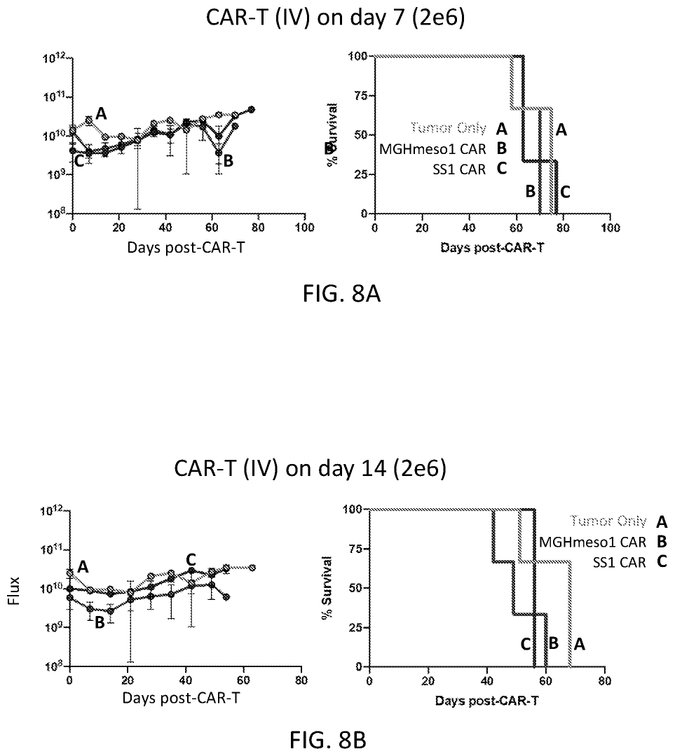

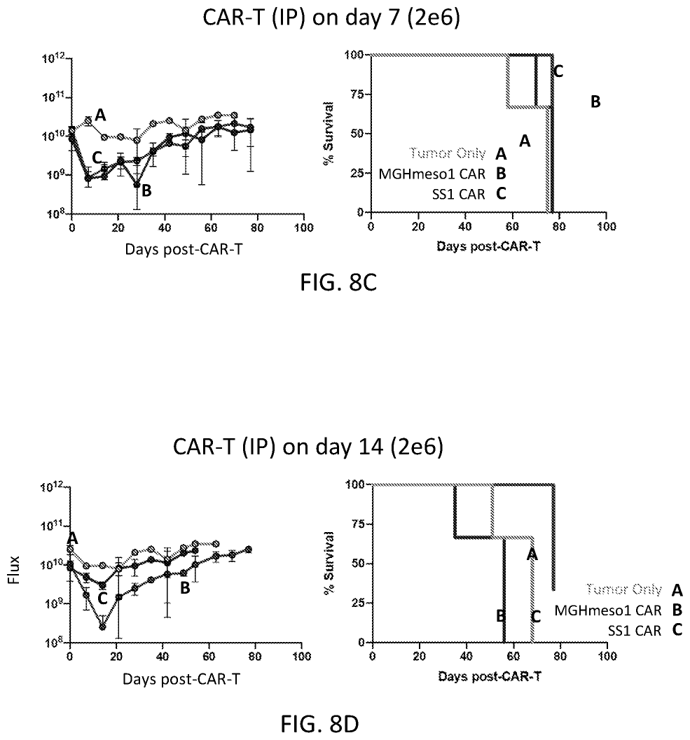

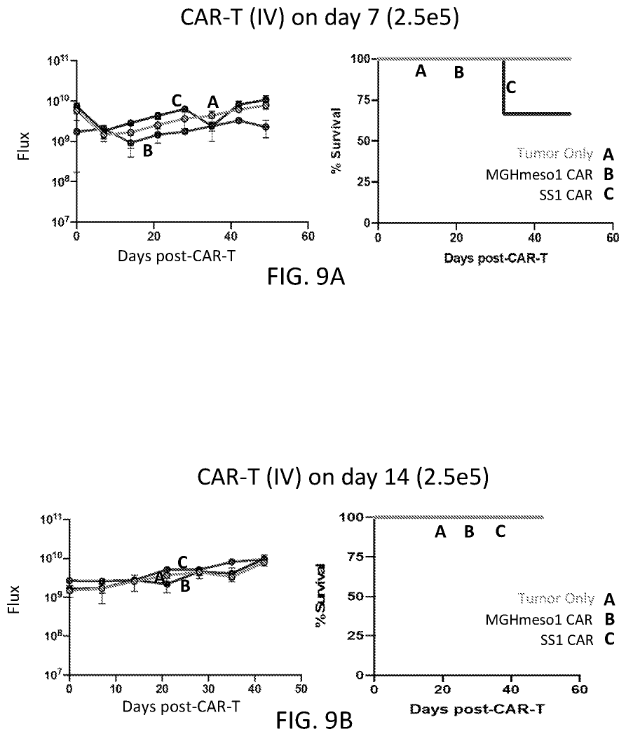

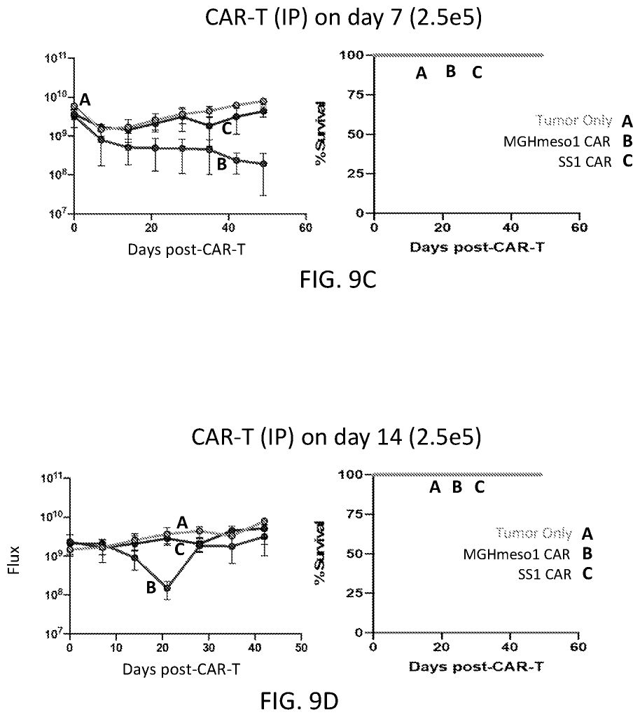

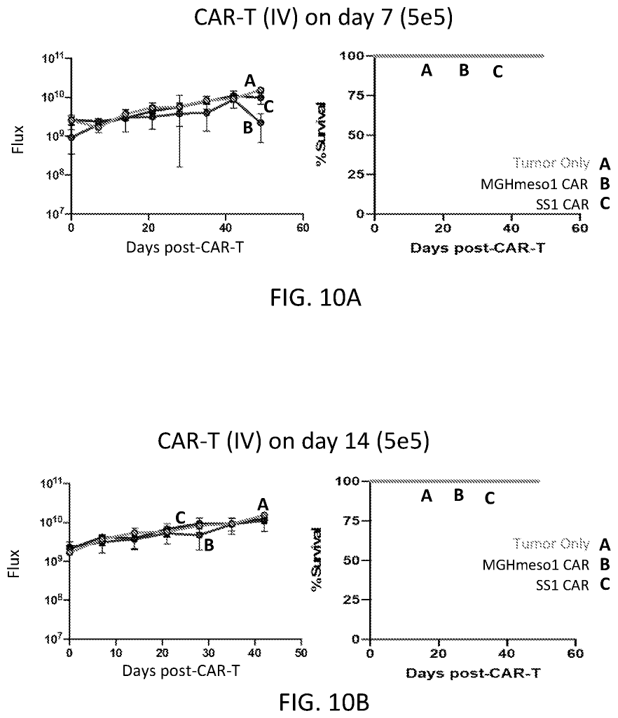

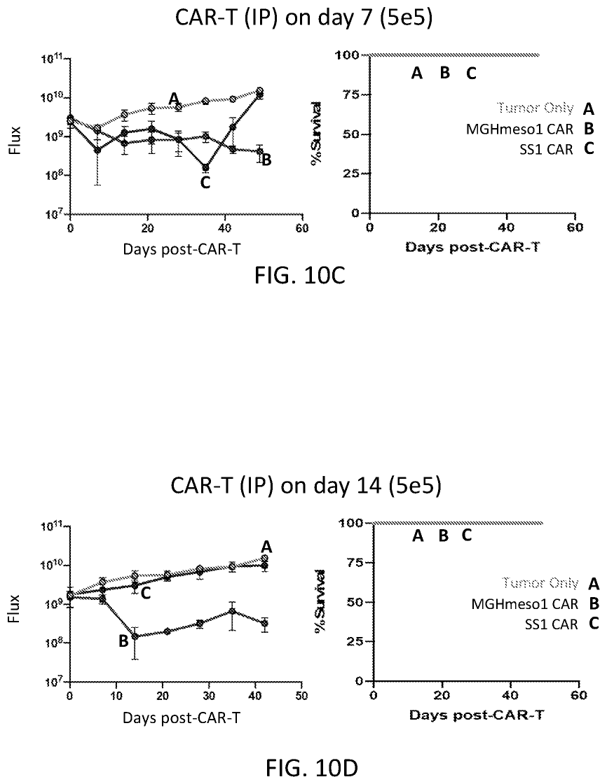

FIGS. 8A-8D compare the flux and percent survival of mice injected with PANC1 pancreatic cancer cells and administered SS1-BBz or MGHmesol L/H-BBz CAR T cells at a dose of 2e6 CAR T cells. Results from FIGS. 8A-8D show that MGHmesol L/H CAR T cells have increased efficacy when administered 14 days post cancer cell injection compared to SS1 CAR T cells when administered by IP injection to mice with PANC1 cancer. These results further demonstrate the potential utility and advantage of MGHmesol CAR T cells for treating a mesothelin-expressing cancer and that both timing of administration as well as the mode of administration of said CAR T cells may be a factor in the relative efficacy of treatment. A series of experiments were performed to assay optimal dosing, timing, and administration routes for CAR T cells (FIGS. 9A-10D). FIGS. 9A-9D compare the flux and percent survival of mice injected with a different line of pancreatic cancer cells (CAPAN2) and administered SS1-BBz or MGHmesol L/H-BBz CAR T cells at a dose of 2.5e5 CAR T cells. Results from FIGS. 9A-9D show that SS1 CAR T cells and MGHmesol CAR T cells have similar efficacy at treating CAPAN2 cancer in mice. FIGS. 10A-10D compare the flux and percent survival of mice injected with CAPAN2 cells and administered SS1-BBz or MGHmesol L/H-BBz CAR T cells at a dose of 5e5 CAR T cells. Results from FIGS. 10A-10D show that SS1 CAR T cells and MGHmesol CAR T cells have similar efficacy at treating CAPAN2 cancer in mice.

Overall, these results demonstrate the utility of MGHmesol CAR T cells for treating mesothelin-expressing cancers, in particular pancreatic cancers, suggest that MGHmesol CAR-T administration via IP is more effective than IV, and suggest MGHmesol CAR T cells may be more potent than SS1 depending upon the timing and mode of administration. In addition, the results suggest MGHmesol CAR T therapy may be more effective at a lower dose than that required for SS1 CAR T. Results also suggest that that 2.5e5 tumor cells can engraft in mice; millions are not needed.

### Example 2: Anti-Mesothelin CAR-T Cells with TEAM FAP

CAR T cells have revolutionized the treatment of hematological malignancies. However, targeting solid cancer types has proven to be more difficult due to their heterogenous target antigen expression, antigen escape of a single targeted antigen and the tumor microenvironment. To address these challenges, a chimeric antigen receptor (CAR) T cell directed towards mesothelin secreting a T cell engaging molecule (TEAM) against fibroblast activation protein (FAP) that redirects CAR T cells and is able to recruit bystander T cells towards FAP was designed. This allows targeting of the stroma and tumor microenvironment rather than just the tumor cells themselves.

Mesothelin has been associated with tumor invasion and is highly expressed on various cancer types such as mesothelioma, pancreatic, pleural, breast and ovarian cancer as well as other types of cancer. Fibroblast activation protein (FAP) is distributed in the extracellular matrix (ECM) being expressed and deposited by cancer associated fibroblasts (CAFs). CAFs have been implicated with different tumor promoting functions. Using TEAM targeting FAP allows to target CAFs and the ECM by itself.

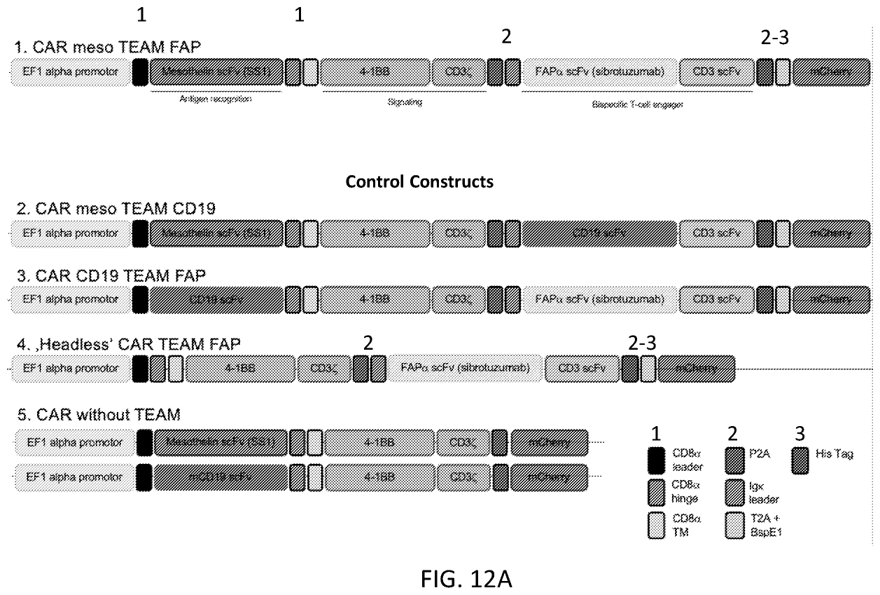

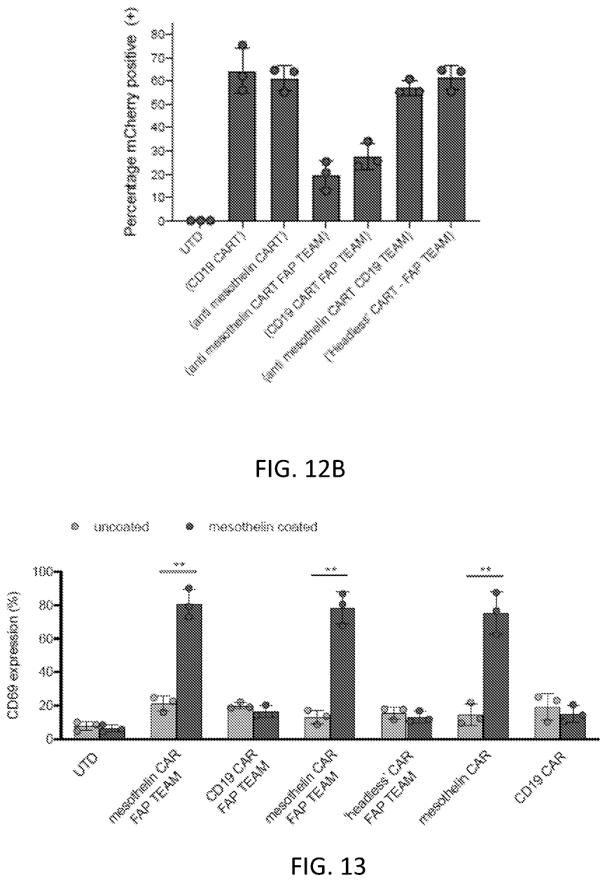

Anti-mesothelin CAR T constructs were designed to include SS1 anti-mesothelin CAR of Example 1, a TEAM comprising the FAP scFv antibody reagent sibrotuzumab, and a CD3 scFv antibody reagent (referred to herein as the anti-mesothelin CAR FAP TEAM) (FIG. 12A). The SS1 anti-mesothelin CAR FAP TEAM showed efficient transduction into donor T cells (FIG. 12B); a median transduction efficiency of 19.6% was achieved for anti-mesothelin CAR FAP TEAM. To determine potential efficacy, SS1 anti-mesothelin CAR FAP TEAM T cell activation was quantified using a CD69 activation assay. Results showed that SS1 anti-mesothelin CAR FAP Team CAR T cells were activated when contacted with mesothelin (FIG. 13) indicating that the CAR is operating correctly (n=3 individual donors).

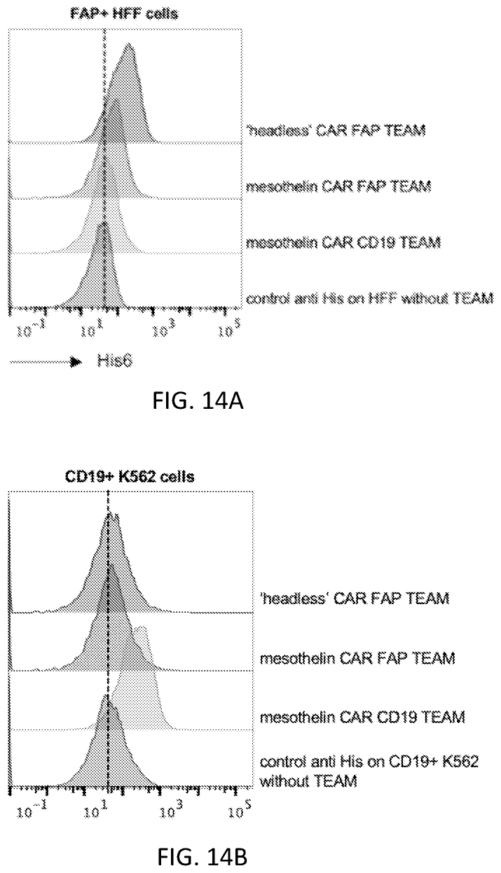

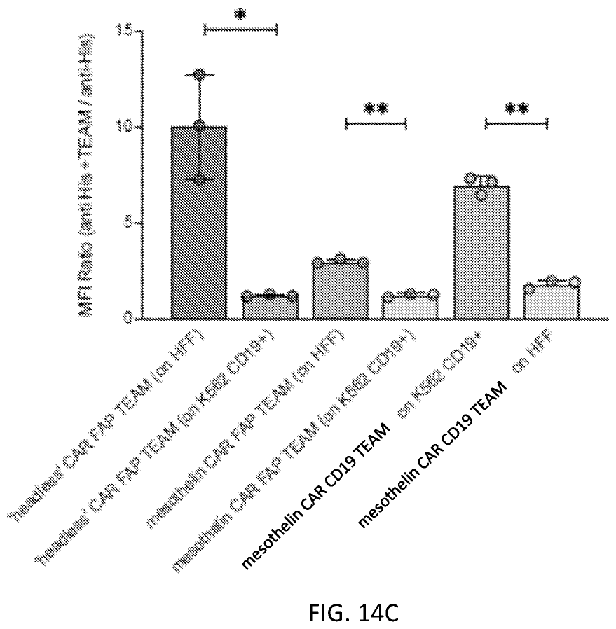

SS1 anti-mesothelin CAR FAP TEAM T cells were also shown to efficiently secrete FAP. Cells expressing FAP (Human foreskin fibroblasts (HFF) cells) or CD19 (K562 Cells, negative control) were treated with supernatant from CAR FAP TEAM T cell culture (FIGS. 14A-14C). Results shows that HFF cells were significantly bound by FAP TEAM (FIG. 14A, 14C), whereas K562 cells were not. FAP binding was detected using an anti-His antibody that bound to the TEAM. These results suggest: 1) FAP TEAM is properly produced and secreted, and 2) FAP TEAM is specific for FAP expressing cells.

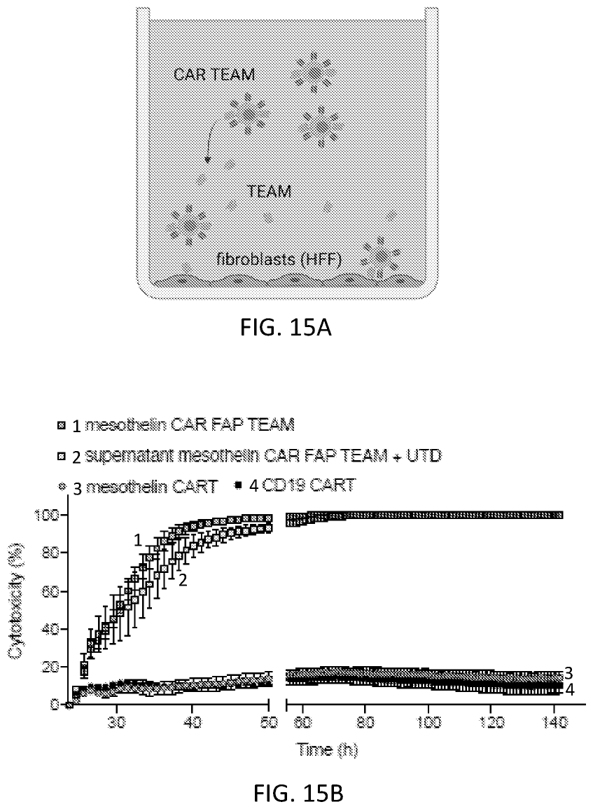

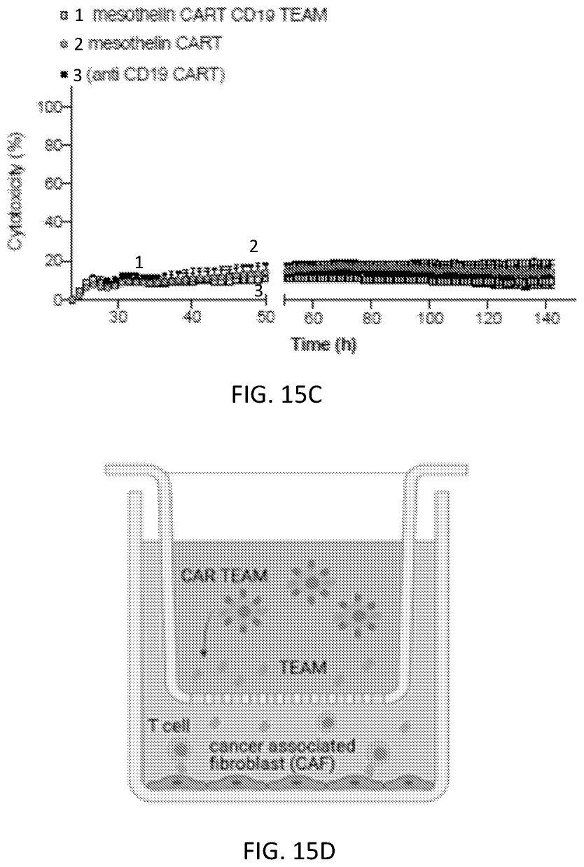

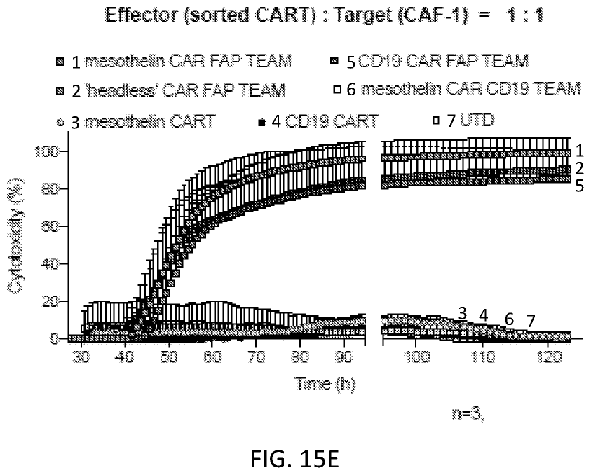

The SS1 anti-mesothelin CAR FAP TEAM T cells were cytotoxic against HFF cells in cell culture (FIG. 15A-15B n=3 individual T-cell donors, each donor in duplicate). In contrast, SS1 anti-mesothelin CAR CD19 TEAM T cells did not show toxicity against HFF cells suggesting that the FAP TEAM significantly contributes to cytotoxicity. In a co-culture system using an impedance-based cytotoxicity assay, tumor lysis through bystander T cells and the redirection of CART cells could be observed. Impedance assays using divided cell cultures suggest that secretion of the FAP TEAM is sufficient to produce activate bystander T cells and produce cytotoxicity against cancer associated fibroblasts (CAF) absent physical interaction with of the SS1 anti-mesothelin CAR FAP TEAM T cells (FIG. 15E, n=3 individual T cell donors, each donor in duplicate). However, physical interaction of the CAR FAP TEAM T cell with the cancer may increase the rate at which cancer cells are killed (FIGS. 15A, 15E).

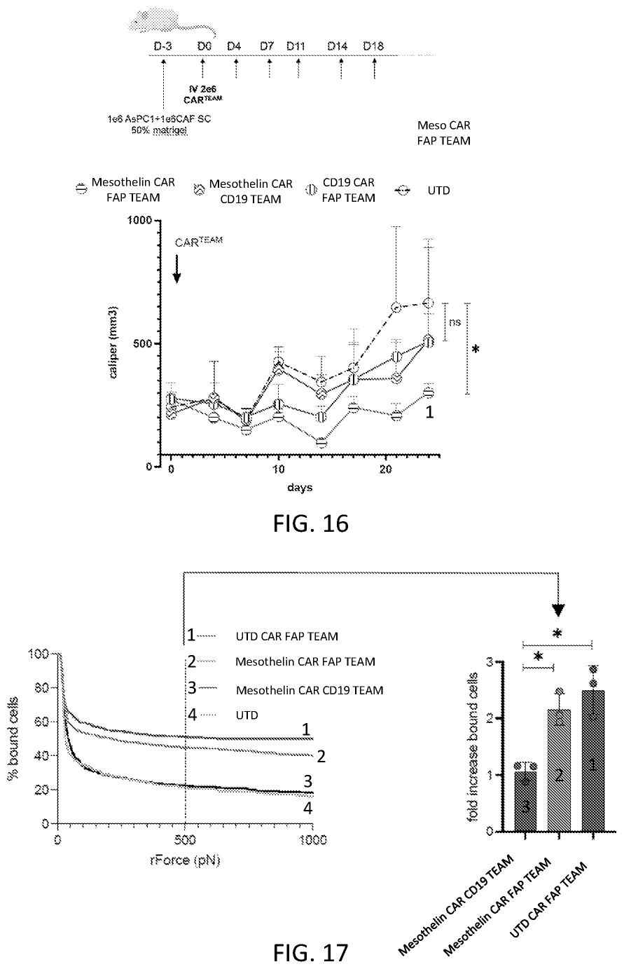

The SS1 anti-mesothelin CAR FAP TEAM also decreased pancreatic cancer proliferation in vivo. Experiments were performed in a murine model of subcutaneously implanted pancreatic cancer cell line admixed with cancer associated fibroblasts (CAFs), the mixture of which mimics pancreatic cancer and tumor microenvironment (TME). Injection with anti-mesothelin CAR FAP TEAM cells resulted in a superior tumor control in comparison to control constructs and untransduced T cells (UTDs) (FIG. 16). These results demonstrate the efficacy of the anti-mesothelin CAR FAP TEAM in treating a mesothelin expressing cancer in combination with a TME and that efficacy of the anti-mesothelin CAR FAP TEAM extends to in vivo use.

Next, the strength (e.g., avidity) of the FAP TEAM mediated physical interaction between FAP expressing fibroblasts and CAR T cells was quantified using acoustic microscopy. HFF (human foreskin fibroblasts) were attached on poly-L-lysine-coated microfluidic chips for at least 3 hours prior to testing on the z-Movi Cell Avidity Analyzer (Lumicks). CellTrace far-red labeled (ThermoFisher Scientific) flow cytometric sorted CAR T-cells or untransduced T cells (UTD) incubated with isolated TEAM molecules were bound for 5 minutes prior to ramping up acoustic force. Experiments were performed with two different CAR T-cell constructs, including UTDs co-incubated with TEAM molecules generated from 3 normal donors. Results showed that FAP-specific TEAM molecules not only increase the avidity of anti-mesothelin CAR FAP TEAM cells relative to UTD, but also of UTDs co-incubated with FAP-specific molecules (FIG. 17). These results suggest that the FAP TEAM can recruit T cells independent of the presence of a CAR and can be broadly useful to target immune system activity to the TME.

Overall, these results demonstrate that adding secreted TEAMS to CAR T cells opens two additional therapeutic avenues. First, to target not only the extracellular matrix but also the extracellular matrix generating cancer associated fibroblasts. Second, to recruit and redirect CART cells and bystander T cells. Based on these results, the CART cells secreting a TEAM that targets the extracellular matrix might generally increase the efficacy of CART cells in solid tumors.

What is claimed is:

1. A chimeric antigen receptor (CAR) T cell comprising a heterologous nucleic acid molecule, wherein the heterologous nucleic acid molecule comprises:
(a) a first polynucleotide encoding a CAR comprising: an extracellular antigen-binding domain that binds to a tumor antigen comprising mesothelin or a portion thereof; a transmembrane domain; and an intracellular signaling domain.

2. The CAR T cell of claim 1, further comprising:
(b) a second polynucleotide encoding a therapeutic agent.

3. The CAR T cell of claim 2, wherein the second polynucleotide is part of the heterologous nucleic acid molecule.

4. The CAR T cell of claim 2, further comprising a second heterologous nucleic acid molecule comprising the second polynucleotide.

5. The CAR T cell of any of claims 1-4, wherein the therapeutic agent comprises an antibody reagent.

6. The CAR T cell of claim 5, wherein the antibody reagent comprises a single chain antibody or a single domain antibody.

7. The CAR T cell of claim 5, wherein the antibody reagent comprises a bispecific antibody reagent.

8. The CAR T cell of claim 7, wherein the bispecific antibody reagent comprises a T cell engaging antibody molecule (TEAM).

9. The CAR T cell of claim 6, wherein the single domain antibody comprises a camelid antibody.

10. The CAR T cell of any of claims 2-9, wherein the therapeutic agent comprises a cytokine.

11. The CAR T cell of any one of claims 2-10, wherein the CAR and the therapeutic agent are produced in the form of a single polypeptide, which is cleaved to generate separate CAR and therapeutic agent molecules.

12. The CAR T cell of claim 11, wherein the single polypeptide comprises a cleavable moiety between the CAR and the therapeutic agent.

13. The CAR T cell of claim 12, wherein the cleavable moiety comprises: a 2A peptide, a 2A ribosomal skip sequence, or an IRES.

14. The CAR T cell of claim 13, wherein the 2A peptide comprises P2A or T2A.

15. The CAR T cell of any one of claims 2-14, wherein the CAR and the therapeutic agent are each constitutively expressed.

16. The CAR T cell of any one of claims 2-15, wherein expression of the CAR and the therapeutic agent is driven by an elongation factor-1 alpha (EF1α) promoter.

17. The CAR T cell of any one of claims 2-14, wherein the therapeutic agent is expressed under the control of an inducible promoter, which is optionally inducible by T cell receptor or CAR signaling.

18. The CAR T cell of claim 17, wherein the inducible promoter comprises the NFAT promoter.

19. The CAR T cell of any one of claims 2-18, wherein the CAR is expressed under the control of a constitutive promoter and the therapeutic agent is expressed under the control of an inducible promoter, which is optionally inducible by T cell receptor or CAR signaling.

20. The CAR T cell of any one of claims 2-19, wherein the CAR expressed from a first vector and the therapeutic agent is expressed from a second vector.

21. The CAR T cell of any one of claims 1-20, wherein the CAR further comprises one or more co-stimulatory domains.

22. The CAR T cell of any one of claims 1-21, wherein the antigen-binding domain of the CAR comprises an antibody, a single chain antibody, a single domain antibody, or a ligand.

23. The CAR T cell of any one of claims 1-22, wherein the transmembrane domain of the CAR comprises a CD8 hinge/transmembrane domain, which optionally comprises the sequence of SEQ ID NO: 24, or a variant thereof.

24. The CAR T cell of any one of claims 1-23, wherein the intracellular signaling domain comprises a CD35 intracellular signaling domain, which optionally comprises the sequence of any one of SEQ ID NOs: 26-27, or a variant thereof.

25. The CAR T cell of any one of claims 1-24, wherein the co-stimulatory domain comprises a 4-1BB co-stimulatory domain, which optionally comprises the sequence of SEQ ID NO: 25 or a variant thereof.

26. The CAR T cell of any one of claims 2-25, wherein the therapeutic agent binds to a tumor antigen.

27. The CAR T cell of claim 26, wherein the tumor antigen to which the therapeutic agent binds is a solid tumor antigen.

28. The CAR T cell of claim 26, wherein the tumor antigen to which the therapeutic agent binds is an activated fibroblast marker.

29. The CAR T cell of claim 28, wherein the activated fibroblast marker comprises: αSMA (ACTA2), fibroblast activation protein (FAP), platelet derived growth factor receptor-α and -β(PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1).

30. The CAR T cell of claim 29, wherein the activated fibroblast marker comprises FAP.

31. The CAR T cell of any one of claims 26-30, wherein the VH of the tumor antigen binding domain of the antibody reagent comprises an amino acid sequence of SEQ ID NO: 6, or an amino acid sequence having at least 90% sequence identity to the amino acid sequence of SEQ ID NO: 6.

32. The CAR T cell any one of claims 26-31, wherein the VL of the tumor antigen binding domain of the antibody reagent comprises an amino acid sequence of SEQ ID NO: 7, or an amino acid sequence having at least 90% sequence identity to the amino acid sequence of SEQ ID NO: 7.

33. The CAR T cell any one of claims 26-32, wherein the tumor antigen binding domain of the antibody reagent comprises an amino acid sequence of SEQ ID NO: 8, or an amino acid sequence having at least 90% sequence identity to the amino acid sequence of SEQ ID NO: 8.

34. The CAR T cell of any one of claims 26-32, wherein the VH of the tumor antigen binding domain of the antibody reagent is N-terminal to the VL of the tumor antigen binding domain of the antibody reagent.

35. The CAR T cell any one of claims 26-32, wherein the VL of the tumor antigen binding domain of the antibody reagent is N-terminal to the VH of the tumor antigen binding domain of the antibody reagent.

36. The CAR T cell of any one of claims 31-35, wherein the tumor antigen binding domain of the antibody reagent comprises a single-chain variable fragment (scFv) or a single domain antibody, which optionally comprises a sequence of SEQ ID NO: 8, or a variant thereof.

37. The CAR T cell of any one of claims 5-36, wherein the antibody reagent binds to a T cell surface antigen.

38. The CAR T cell of claim 37, wherein the T cell surface antigen comprises T cell receptor (TCR), a TCR complex component, or a portion of either thereof.

39. The CAR T cell of claim 38, wherein the TCR complex component is chosen from CD3 (e.g., the CD3ε subunit, the CD3ξ subunit, or the CD3γ subunit), the CD4 coreceptor, and the CD8 coreceptor.

40. The CAR T cell of claim 39, wherein the TCR complex component is CD3.

41. The CAR T cell of any one of claims 37-40, wherein the VH of the T cell surface antigen-binding domain of the antibody reagent comprises an amino acid sequence of SEQ ID NO: 9, or an amino acid sequence having at least 90% sequence identity to the amino acid sequence of SEQ ID NO: 9.

42. The CAR T cell of any one of claims 37-41, wherein the VL of the T cell surface antigen-binding domain of the antibody reagent comprises an amino acid sequence of SEQ ID NO: 10, or an amino acid sequence having at least 90% sequence identity to the amino acid sequence of SEQ ID NO: 10.

43. The CAR T cell of any one of claims 37-42, wherein the VH of the T cell surface antigen-binding domain of the antibody reagent is N-terminal to the VL of the T cell surface antigen-binding domain of the antibody reagent.

44. The CAR T cell of any of claims 37-42, wherein the VL of the T cell surface antigen-binding domain of the antibody reagent is N-terminal to the VH of the T cell surface antigen-binding domain of the antibody reagent.

45. The CAR T cell of any one of claims 37-42, wherein the T cell surface antigen-binding domain of the antibody reagent comprises a scFv or a single domain antibody, which optionally comprises a sequence of any one of SEQ ID NOs: 11-12, or a variant thereof.

46. The CAR T cell of any one of claims 1-45, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 13, or an amino acid sequence with no more than 1, 2, or 3 amino acid substitutions of SEQ ID NO: 13; the CDR-H2 comprises an amino acid sequence of SEQ ID NO: 14, or an amino acid sequence with no more than 1, 2, or 3 amino acid substitutions of SEQ ID NO: 14; and the CDR-H3 comprises an amino acid sequence of SEQ ID NO: 15, or an amino acid sequence with no more than 1, 2, or 3 amino acid substitutions of SEQ ID NO: 15, and
(b) a light chain variable domain (VL) comprising three complementarity determining regions CDR-L1, CDR-L2, and CDR-L3, wherein the CDR-L1 comprises an amino acid sequence of SEQ ID NO: 16, or an amino acid sequence with no more than 1, 2, or 3 amino acid substitutions of SEQ ID NO: 16; the CDR-L2 comprises an amino acid sequence of SEQ ID NO: 17, or an amino acid sequence with no more than 1, 2, or 3 amino acid substitutions of SEQ ID NO: 17; and the CDR-L3 comprises an amino acid sequence of SEQ ID NO: 18, or an amino acid sequence with no more than 1, 2, or 3 amino acid substitutions of SEQ ID NO: 18.

47. The CAR T cell of claim 46, wherein the VH of the antigen-binding domain of the CAR comprises an amino acid sequence of SEQ ID NO: 19, or an amino acid sequence having at least 90% sequence identity to the amino acid sequence of SEQ ID NO: 19.

48. The CAR T cell of claim 46 or 47, wherein the VL of the antigen-binding domain of the CAR comprises an amino acid sequence of SEQ ID NO: 20, or an amino acid sequence having at least 90% sequence identity to the amino acid sequence of SEQ ID NO: 20.

49. The CAR T cell of any of claims 46-48, wherein the VH of the antigen-binding domain of the CAR is N-terminal to the VL of the antigen-binding domain of the CAR.

50. The CAR T cell of any of claims 46-48, wherein the VL of the antigen-binding domain of the CAR is N-terminal to the VH of the antigen-binding domain of the CAR.

51. The CAR T cell of any one of claims 46-48, wherein the antigen-binding domain of the CAR comprises a scFv or a single domain antibody, which optionally comprises a sequence selected from the group consisting of SEQ ID NOs: 21 or 22, and variants thereof.

52. The CAR T cell of any one of claims 1-45, wherein the antigen-binding domain of the CAR comprises:
(a) a heavy chain variable domain (VH) comprising complementarity determining regions CDR-H1, CDR-H2, and CDR-H3, wherein the CDR-H1 comprises the amino acid sequence of SEQ ID NO: 42, or an amino acid sequence with no more than 1, 2, or 3 amino acid substitutions relative thereto; the CDR-H2 comprises an amino acid sequence of SEQ ID NO: 43, or an amino acid sequence with no more than 1, 2, or 3 amino acid substitutions relative thereto; and the CDR-H3 comprises an amino acid sequence of SEQ ID NO: 44, or an amino acid sequence with no more than 1, 2, or 3 amino acid substitutions relative thereto, and
(b) a light chain variable domain (VL) comprising complementarity determining regions CDR-L1, CDR-L2, and CDR-L3, wherein the CDR-L1 comprises the amino acid sequence of SEQ ID NO: 45, or an amino acid sequence with no more than 1, 2, or 3 amino acid substitutions relative thereto; the CDR-L2 comprises an amino acid sequence of SEQ ID NO: 46, or an amino acid sequence with no more than 1, 2, or 3 amino acid substitutions relative thereto; and the CDR-L3 comprises an amino acid sequence of SEQ ID NO: 47, or an amino acid sequence with no more than 1, 2, or 3 amino acid substitutions relative thereto.

53. The CAR T cell of claim 52, wherein the VH of the antigen-binding domain of the CAR comprises the SS1 heavy chain sequence of SEQ ID NO: 40, or an amino acid sequence having at least 90% sequence identity to the SS1 heavy chain sequence of SEQ ID NO: 40.

54. The CAR T cell of claim 52 or of claim 53, wherein the VL of the antigen-binding domain of the CAR comprises the SS1 light chain sequence of SEQ ID NO: 41, or an amino acid sequence having at least 90% sequence identity to the SS1 light chain sequence of SEQ ID NO: 41.

55. The CAR T cell of any one of claims 52-54, wherein the VH of the antigen-binding domain of the CAR is N-terminal to the VL of the antigen-binding domain of the CAR.

56. The CAR T cell of any one of claims 52-54, wherein the VL of the antigen-binding domain of the CAR is N-terminal to the VH of the antigen-binding domain of the CAR.

57. The CAR T cell of any one of claims 52-54, wherein the antigen-binding domain of the CAR comprises a scFv or a single domain antibody, which optionally comprises the SS1 VH amino acid sequence of SEQ ID NO: 40 and the SS1 VL amino acid sequence of SEQ ID NO: 41, and variants thereof.

58. The CAR T cell of any one of claims 1-57, wherein the heterologous nucleic acid molecule further comprises a suicide gene.

59. A chimeric antigen receptor (CAR) comprising:
(a) an extracellular antigen-binding domain that binds to a tumor antigen comprising mesothelin or a portion thereof,
(b) a transmembrane domain, and
(c) an intracellular signaling domain.

60. The CAR of claim 59, further comprising a polynucleotide or polypeptide therapeutic agent.

61. The CAR of either of claim 59 or 60, wherein the therapeutic agent comprises an antibody reagent.

62. The CAR of claim 61, wherein the antibody reagent comprises a single chain antibody or a single domain antibody.

63. The CAR of claim 62, wherein the antibody reagent comprises a bispecific antibody reagent.

64. The CAR of claim 63, wherein the bispecific antibody reagent comprises a T cell engaging molecule (TEAM).

65. The CAR of claim 62, wherein the single domain antibody comprises a camelid antibody.

66. The CAR of any of claims 60-65, wherein the therapeutic agent comprises a cytokine.

67. The CAR of any one of claims 60-66, wherein the CAR and the therapeutic agent are produced in the form of a single polypeptide, which is cleaved to generate separate CAR and therapeutic agent molecules.

68. The CAR of claim 67, wherein the single polypeptide comprises a cleavable moiety between the CAR and the therapeutic agent.

69. The CAR of claim 68, wherein the cleavable moiety comprises a 2A peptide.

70. The CAR of claim 69, wherein the 2A peptide comprises P2A or T2A.

71. The CAR of any one of claims 60-70, wherein the CAR and the therapeutic agent are constitutively expressed.

72. The CAR of any one of claims 60-71, wherein expression of the CAR and the therapeutic agent is driven by an elongation factor-1 alpha (EFla) promoter.

73. The CAR of any one of claims 60-70, wherein expression of the CAR and the therapeutic agent is driven an inducible promoter, which is optionally inducible by T cell receptor or CAR signaling.

74. The CAR of claim 73, wherein the inducible promoter comprises the NFAT promoter.

75. The CAR of any one of claims 60-70, wherein the CAR is expressed under the control of a constitutive promoter and the therapeutic agent is expressed under the control of an inducible promoter, which is optionally inducible by T cell receptor or CAR signaling.

76. The CAR of any one of claims 59-75, wherein the CAR further comprises one or more co-stimulatory domains.

77. The CAR of any one of claims 59-76, wherein the antigen-binding domain of the CAR comprises an antibody, a single chain antibody, a single domain antibody, or a ligand.

78. The CAR of any one of claims 59-77, wherein the transmembrane domain of the CAR comprises a CD8 hinge/transmembrane domain, which optionally comprises the sequence of SEQ ID NO: 24, or a variant thereof.

79. The CAR of any one of claims 59-78, wherein the intracellular signaling domain comprises a CD3ξ intracellular signaling domain, which optionally comprises the sequence of SEQ ID NO: 26-27, or a variant thereof.

80. The CAR of any one of claims 59-79, wherein the co-stimulatory domain comprises a 4-1BB co-stimulatory domain, which optionally comprises the sequence of any one of SEQ ID NOs: 25, or a variant thereof.

81. The CAR of any one of claims 60-80, wherein the therapeutic agent binds to a tumor antigen.

82. The CAR of claim 81, wherein the tumor antigen to which the therapeutic agent binds is a solid tumor antigen.

83. The CAR of claim 81, wherein the tumor antigen to which the therapeutic agent binds is an activated fibroblast marker.

84. The CAR of claim 83, wherein the activated fibroblast marker comprises: αSMA (ACTA2), fibroblast activation protein (FAP), platelet derived growth factor receptor-α and -β (PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1).

85. The CAR of claim 84, wherein the activated fibroblast marker comprises FAP.

86. The CAR of any one of claims 81-85, wherein the VH of the tumor antigen binding domain of the antibody reagent comprises an amino acid sequence of SEQ ID NO: 6, or an amino acid sequence having at least 90% sequence identity to the amino acid sequence of SEQ ID NO: 6.

87. The CAR of any one of claims 81-86, wherein the VL of the tumor antigen binding domain of the antibody reagent comprises an amino acid sequence of SEQ ID NO: 7, or an amino acid sequence having at least 90% sequence identity to the amino acid sequence of SEQ ID NO: 7.

88. The CAR of either of claim 86 or 87, wherein the VH of the tumor antigen binding domain of the antibody reagent is N-terminal to the VL of the tumor antigen binding domain of the antibody reagent.

89. The CAR of either of claim 86 or 87, wherein the VL of the tumor antigen binding domain of the antibody reagent is N-terminal to the VH of the tumor antigen binding domain of the antibody reagent.

90. The CAR of any one of claims 81-89, wherein the tumor antigen binding domain of the antibody reagent comprises a single-chain variable fragment (scFv) or a single domain antibody, which optionally comprises a sequence of SEQ ID NO: 21 or 22, or a variant thereof.

91. The CAR of any one of claims 61-90, wherein the antibody reagent binds to a T cell surface antigen.

92. The CAR of claim 91, wherein the T cell surface antigen comprises T cell receptor (TCR), a TCR complex component, or a portion of either thereof.

93. The CAR of claim 92, wherein the TCR complex component is chosen from CD3 (e.g., the CD3εsubunit, the CD3ζ subunit, or the CD3γ subunit), the CD4 coreceptor, and the CD8 coreceptor.

94. The CAR of claim 93, wherein the TCR complex component is CD3.

95. The CAR of any one of claims 91-94, wherein the VH of the T cell surface antigen-binding domain of the antibody reagent comprises an amino acid sequence of SEQ ID NO: 9, or an amino acid sequence having at least 90% sequence identity to the amino acid sequence of SEQ ID NO: 9.

96. The CAR of claim any one of claims 91-95, wherein the VL of the T cell surface antigen-binding domain of the antibody reagent comprises an amino acid sequence of SEQ ID NO: 10, or an amino acid sequence having at least 90% sequence identity to the amino acid sequence of SEQ ID NO: 10.

97. The CAR of any of claims 91-96, wherein the VH of the T cell surface antigen-binding domain of the antibody reagent is N-terminal to the VL of the T cell surface antigen-binding domain of the antibody reagent.

98. The CAR of any of claims 91-96, wherein the VL of the T cell surface antigen-binding domain of the antibody reagent is N-terminal to the VH of the T cell surface antigen-binding domain of the antibody reagent.

99. The CAR of any one of claims 91-96, wherein the T cell surface antigen-binding domain of the antibody reagent comprises a scFv or a single domain antibody, which optionally comprises a sequence of SEQ ID NO:11-12, or a variant thereof.

100. The CAR of any one of claims 59-99, wherein the antigen-binding domain of the CAR comprises:
(a) a heavy chain variable domain (VH) comprising three complementarity determining regions CDR-H1, CDR-H2, and CDR-H3, wherein the CDR-H1 comprises an amino acid sequence of SEQ ID NO: 13, or an amino acid sequence with no more than 1, 2, or 3 amino acid substitutions of SEQ ID NO: 13; the CDR-H2 comprises an amino acid sequence of SEQ ID NO: 14, or an amino acid sequence with no more than 1, 2, or 3 amino acid substitutions of SEQ ID NO: 14; and the CDR-H3 comprises an amino acid sequence of SEQ ID NO: 15, or an amino acid sequence with no more than 1, 2, or 3 amino acid substitutions of SEQ ID NO: 15, and
(b) a light chain variable domain (VL) comprising three complementarity determining regions CDR-L1, CDR-L2, and CDR-L3, wherein the CDR-L1 comprises an amino acid sequence of SEQ ID NO: 16, or an amino acid sequence with no more than 1, 2, or 3 amino acid substitutions of SEQ ID NO: 16; the CDR-L2 comprises an amino acid sequence of SEQ ID NO: 17, or an amino acid sequence with no more than 1, 2, or 3 amino acid substitutions of SEQ ID NO: 17; and the CDR-L3 comprises an amino acid sequence of SEQ ID NO: 18, or an amino acid sequence with no more than 1, 2, or 3 amino acid substitutions of SEQ ID NO: 18.

101. The CAR of claim 100, wherein the VH of the antigen-binding domain of the CAR comprises an amino acid sequence of SEQ ID NO: 19, or an amino acid sequence having at least 90% sequence identity to the amino acid sequence of SEQ ID NO: 19.

102. The CAR of either of claim 100 or 101, wherein the VL of the antigen-binding domain of the CAR comprises an amino acid sequence of SEQ ID NO: 20, or an amino acid sequence having at least 90% sequence identity to the amino acid sequence of SEQ ID NO: 20.

103. The CAR of any of claims 100-102, wherein the VH of the antigen-binding domain of the CAR is N-terminal to the VL of the antigen-binding domain of the CAR.

104. The CAR of any of claims 100-102, wherein the VL of the antigen-binding domain of the CAR is N-terminal to the VH of the antigen-binding domain of the CAR.

105. The CAR of any one of claims 100-102, wherein the antigen-binding domain of the CAR comprises a scFv or a single domain antibody, which optionally comprises a sequence selected from the group consisting of SEQ ID NOs: 21 or 22, and variants thereof.

106. The CAR of any one of claims 100-102, comprising an amino acid sequence having at least 90% sequence identity to the amino acid sequence of SEQ ID NO: 21 or 22.

107. The CAR of claim 106, comprising an amino acid sequence of SEQ ID NO: 21 or 22.

108. The CAR of any one of claims 59-99, wherein the antigen-binding domain of the CAR comprises:
(a) a heavy chain variable domain (VH) comprising complementarity determining regions CDR-H1, CDR-H2, and CDR-H3, wherein the CDR-H1 comprises the amino acid sequence of SEQ ID NO: 42, or an amino acid sequence with no more than 1, 2, or 3 amino acid substitutions relative thereto; the CDR-H2 comprises an amino acid sequence of SEQ ID NO: 43, or an amino acid sequence with no more than 1, 2, or 3 amino acid substitutions relative thereto; and the CDR-H3 comprises an amino acid sequence of SEQ ID NO: 44, or an amino acid sequence with no more than 1, 2, or 3 amino acid substitutions relative thereto, and
(b) a light chain variable domain (VL) comprising complementarity determining regions CDR-L1, CDR-L2, and CDR-L3, wherein the CDR-L1 comprises the amino acid sequence of SEQ ID NO: 45, or an amino acid sequence with no more than 1, 2, or 3 amino acid substitutions relative thereto; the CDR-L2 comprises an amino acid sequence of SEQ ID NO: 46, or an amino acid sequence with no more than 1, 2, or 3 amino acid substitutions relative thereto; and the CDR-L3 comprises an amino acid sequence of SEQ ID NO: 47, or an amino acid sequence with no more than 1, 2, or 3 amino acid substitutions relative thereto.

109. The CAR of claim 108, wherein the VH of the antigen-binding domain of the CAR comprises the SS1 heavy chain sequence of SEQ ID NO: 40, or an amino acid sequence having at least 90% sequence identity to the SS1 heavy chain sequence of SEQ ID NO: 40.

110. The CAR of either one of claim 108 or 109, wherein the VL of the antigen-binding domain of the CAR comprises the SS1 light chain sequence of SEQ ID NO: 41, or an amino acid sequence having at least 90% sequence identity to the SS1 light chain sequence of SEQ ID NO: 41.

111. The CAR of any of claims 108-110, wherein the VH of the antigen-binding domain of the CAR is N-terminal to the VL of the antigen-binding domain of the CAR.

112. The CAR of any of claims 108-110, wherein the VL of the antigen-binding domain of the CAR is N-terminal to the VH of the antigen-binding domain of the CAR.

113. The CAR of any one of claims 108-110, wherein the antigen-binding domain of the CAR comprises a scFv or a single domain antibody, which optionally comprises the SS1 VH amino acid sequence of SEQ ID NO: 40 and the SS1 VL amino acid sequence of SEQ ID NO: 41, and variants thereof.

114. The CAR of any of claims 59-113, comprising an amino acid sequence of any one of SEQ ID NOs: 1-5.

115. The CAR of claim 114, consisting of an amino acid sequence of any one of SEQ ID NOs: 1-5.

116. The CAR of any one of claims 59-114 further comprising a suicide gene.

117. A nucleic acid molecule encoding the CAR polypeptide of any one of claims 59-116.

118. The nucleic acid molecule of claim 117, wherein the nucleic acid molecule is a plasmid or a vector.

119. A viral vector comprising the nucleic acid molecule of claim 117 or 118.

120. The viral vector of claim 119, wherein the viral vector is a lentiviral or adeno-associated viral vector.

121. A CAR polypeptide, or a single polypeptide comprising the CAR polypeptide and the therapeutic agent of any one of claims 1-116.

122. A CAR T cell comprising the CAR of any one of claims 59-116 or the nucleic acid molecule or viral vector of any one of claim 117-120.

123. A pharmaceutical composition comprising the CAR T cell of any one of claim 1-58 or 122, the CAR polypeptide of any one of claim 59-116 or 121, or the nucleic acid molecule or viral vector of any one of claims 117-120.

124. A pharmaceutical composition comprising a therapeutically effective amount of the CAR T cell of any one of claim 1-58 or 122, the CAR polypeptide of any one of claim 59-116 or 121, or the nucleic acid molecule or viral vector of any one of claims 117-120.

125. The pharmaceutical composition of either one of claim 123 or 124 further comprising a pharmaceutically acceptable excipient.

126. A method of treating a patient having cancer, the method comprising administering to the patient a pharmaceutical composition comprising the CAR T cell of any one of claim 1-58 or 122, the CAR polypeptide of any one of claim 59-116 or 121, or the nucleic acid molecule or viral vector of any one of claims 117-120, or the pharmaceutical composition of any one of claims 123-125.

127. The method of claim 126, wherein by targeting the tumor microenvironment, systemic toxicity is reduced.

128. The method of either one of claim 126 or 127, wherein the cancer is characterized by the presence of one or more solid tumors.

129. The method of any of claims 126-128, wherein the cancer is characterized by expression of mesothelin.

130. The method of any of claims 126-129, wherein the tumor microenvironment is characterized by the presence of activated fibroblasts.

131. The method of any of claims 126-130, wherein the tumor microenvironment is characterized by the presence of fibroblast activating protein (FAP).

132. The method of any one of claims 126-131, wherein the cancer is a pancreatic cancer, a lung cancer, an ovarian cancer, endometrial cancer, biliary cancer, gastric cancer, or mesothelioma.

133. A method of treating a patient having cancer, the method comprising administering to the patient a CAR T cell product, genetically modified to secrete a tumor-toxic antibody or cytokine, wherein by directing the cancer toxicity locally to the tumor microenvironment, systemic toxicity is reduced.

134. The method of claim 133, wherein the CAR T cell is genetically modified to deliver an antibody or a bispecific antibody against an activated fibroblast marker to the tumor microenvironment.

135. The method of either of claim 133 or 134, wherein the CAR T cell comprises a polynucleotide encoding a CAR comprising an extracellular antigen-binding domain that binds to a tumor antigen comprising mesothelin or a portion thereof.

136. The method of either one of claim 134 or 135, wherein the bispecific antibody is directed against an activated fibroblast marker and CD3.

137. A method of delivering a therapeutic agent to a tissue or organ in a patient to treat a disease or pathology, the method comprising administering to said patient a CAR T cell, genetically modified to secrete a therapeutic antibody, toxin, or agent, wherein the therapeutic antibody, toxin, or agent would, by itself, be unable to enter or penetrate the tissue or organ.

138. The method of claim 137, wherein the tissue or organ is in the digestive or endocrine system.

139. The method of claim 138, wherein the tissue is in the pancreas or the organ is the pancreas.

140. The method of claim 137, wherein the tissue or organ is in the reproductive system.

141. The method of claim 140, wherein the tissue is in an ovary or the organ is an ovary.

142. The method of claim 137, wherein the tissue or organ is in the respiratory or pulmonary system.

143. The method of claim 142, wherein the tissue is in a lung or the organ is a lung.

144. The method of any one of claims 137-143, wherein the therapeutic antibody is directed against an activated fibroblast marker.

145. The method of any of claims 133-144, wherein the CAR T cell is a CAR T cell of any one of claim 1-58 or 122, or the CAR T cell is comprised within a pharmaceutical composition of any one of claims 123-125.
